(19) United States
(12) Patent Application Publication
(10) Pub. No.: US 2026/0072037 A1
(43) Pub. Date: Mar. 12, 2026

(54) Apparatus and Processes for Magnetic Detection of an Analyte

(71) Applicant: Western Digital Technologies, Inc., San Jose, CA (US)

(72) Inventor: Daniel BEDAU, San Jose, CA (US)

(73) Assignee: Western Digital Technologies, Inc., San Jose, CA (US)

(21) Appl. No.: 18/830,920

(22) Filed: Sep. 11, 2024

(51) Int. Cl. G01N 33/58; C12Q 1/6844

(52) U.S. Cl. CPC G01N 33/587; C12Q 1/6844; G01N 2333/95

(57) ABSTRACT

Embodiments described herein generally relate to new apparatus and processes for magnetically detecting an analyte. In an embodiment is provided an apparatus for magnetically detecting the presence of a target analyte in a sample. The apparatus includes: a plurality of magnetic nanoparticles; and a plurality of magnetic sensors disposed within a fluidic channel, each magnetic sensor of the plurality of magnetic sensors coupled, by a binding link, to at least one magnetic nanoparticle of the plurality of magnetic nanoparticles, the binding link adapted to be disrupted in the presence of a target analyte that breaks a covalent bond of the binding link to release the at least one magnetic nanoparticle from the magnetic sensor and indicate the presence of the target analyte.

### FIELD

Embodiments described herein generally relate to new apparatus and processes for magnetically detecting the presence of an analyte in a sample.

### BACKGROUND

Devices to determine the presence of an analyte, such as chemicals and biologics, in samples are used in health-related diagnostic tools, environmental monitoring, and in various industrial processes such as the manufacture of pharmaceuticals and recycling of solvents. Demands to reduce the time of analysis and increase the sensitivity and selectivity of the devices, even with reduced sample volumes, is a pressing concern.

Conventional technologies for rapid detection of analytes in a sample include a recognition component to interact with the analyte and a detector component to convert the interaction to a measurable signal. Many conventional technologies, however, rely on a tagged analyte binding to the recognition component. For example, immunosensors utilize specific binding affinities of an antibody bound to the device with an antigen (an analyte) present in the sample. Upon binding, a signal, such as a color change or an electrical response, is then output from the device. In addition, many conventional technologies for detecting analytes require a washing stage to prevent false readings caused by association of undesired sample components with the sensor. Further, conventional approaches for detecting analytes do not have in-line detection capabilities, and instead, rely on detection at some point away from the interaction of the analyte with the recognition component. For example, state-of-the-art approaches rely on washing beads (such as magnetic beads) away from a cleavage site and detecting the beads elsewhere. This detection mechanism is problematic for at least the reason that the beads may stick to surfaces of apparatus, potentially leading to false negatives.

There is a need for new apparatus and processes for detecting analytes.

### SUMMARY

Embodiments described herein generally relate to new apparatus and processes for magnetically detecting an analyte. The inventor found a new type of apparatus which includes magnetic nanoparticles (MNPs) coupled to magnetic sensors via binding links. In the presence of an analyte, the binding link becomes disrupted, releasing the MNP from the magnetic sensor. Upon this detachment, or untethering, a signal is immediately output from the apparatus. Advantageously, embodiments of the present disclosure enable detection of different types of chemicals or analytes in a sample. In addition, apparatus may be advantageously factory-calibrated and tested, as the MNPs are already attached to the magnetic sensor during fabrication of the apparatus.

In an embodiment, an apparatus for magnetically detecting the presence of a target analyte in a sample is provided. The apparatus comprises: a plurality of magnetic nanoparticles; and a plurality of magnetic sensors disposed within a fluidic channel, each magnetic sensor of the plurality of magnetic sensors coupled, by a binding link, to at least one magnetic nanoparticle of the plurality of magnetic nanoparticles, the binding link adapted to be disrupted in the presence of a target analyte that breaks a covalent bond of the binding link to release the at least one magnetic nanoparticle from the magnetic sensor and indicate the presence of the target analyte.

Implementations may include one or more of the following. The apparatus may further include processing circuitry configured to detect a signal from the magnetic sensor to indicate the absence of the at least one magnetic nanoparticle when the binding link is disrupted, thereby indicating the presence of the target analyte. The apparatus may be adapted to detect release of the at least one magnetic nanoparticle at the time of the release of the at least one magnetic nanoparticle. At least one binding link may be adapted to be disrupted in the presence of a control analyte present in the sample, the control analyte different from the target analyte. The binding link of the apparatus may be adapted to be disrupted at a second concentration of the target analyte, the second concentration of the target analyte generated from a chemical amplification reaction on the first concentration of the target analyte. The binding link of the apparatus may be adapted to be disrupted in the presence of a species, the species generated from a chemical reaction on the target analyte. The binding link may include a nucleic acid, a polypeptide, a peptidoglycan, or combinations thereof. When the binding link includes the nucleic acid, the target analyte may include an enzyme, a mutagenic chemical, a CRISPR-associated protein, or combinations thereof. When the binding link comprises the polypeptide, the target analyte may include an enzyme, a reactive oxygen species, or combinations thereof. When the binding link comprises the peptidoglycan, the target analyte may include an antibiotic. When the binding link includes the nucleic acid and the target analyte includes the enzyme, the enzyme may include a topoisomerase, a helicase, a polymerase, a nuclease, or combinations thereof. When the binding link comprises the polypeptide and the target analyte comprises the enzyme, the enzyme may include a protease. The binding link may include a carbamate moiety, an amide moiety, an ether moiety, a phosphodiester moiety, or combinations thereof. When the binding link includes the carbamate moiety, the amide moiety, the ether moiety, or combinations thereof, the target analyte may include a transition metal from Group 3 to Group 12 of the periodic table of the elements. When the binding link includes the carbamate moiety, the target analyte may include formaldehyde, a phosphine, or combinations thereof. When the binding link includes the phosphodiester moiety, the target analyte may include a nuclease.

In another embodiment, an apparatus for magnetically detecting the presence of a target analyte in a sample. The apparatus comprises: a plurality of magnetic nanoparticles; and a plurality of magnetic sensors disposed within a fluidic channel, each magnetic sensor of the plurality of magnetic sensors coupled, by a binding link, to at least one magnetic nanoparticle of the plurality of magnetic nanoparticles, the binding link adapted to be disrupted in the presence of a target analyte that breaks a non-covalent bond of the binding link to release the at least one magnetic nanoparticle from the magnetic sensor and indicate the presence of the target analyte.

Implementations may include one or more of the following. The apparatus may further include processing circuitry configured to detect a signal from the magnetic sensor to indicate the absence of the at least one magnetic nanoparticle when the binding link is disrupted, thereby indicating the presence of the target analyte. The apparatus may be adapted to detect release of the at least one magnetic nanoparticle at the time of the release of the at least one magnetic nanoparticle. At least one binding link may be adapted to be disrupted in the presence of a control analyte present in the sample, the control analyte different from the target analyte. The binding link of the apparatus may be adapted to be disrupted at a second concentration of the target analyte, the second concentration of the target analyte generated from a chemical amplification reaction on the first concentration of the target analyte. The binding link of the apparatus may be adapted to be disrupted in the presence of a species, the species generated from a chemical reaction on the target analyte. The binding link may include a lipid, a nonpolar molecule, oil, wax, grease, streptavidin-biotin, a polymer, a salt, or combinations thereof. When the binding link comprises the lipid, the target analyte may include an antibiotic. When the binding link comprises the nonpolar molecule, the oil, the wax, the grease, or combinations thereof, the target analyte may include a surfactant. When the binding link comprises the streptavidin-biotin, the target analyte may include a solvent having a lower polarity than water. When the binding link comprises the polymer, the target analyte may include a solvent that solubilizes the polymer. When the binding link comprises the salt, the target analyte may include an ionic liquid. The binding link may include two or more first nucleic acids non-covalently bonded to one another. The target analyte may include an enzyme, a second nucleic acid, or combinations thereof, the second nucleic acid adapted to displace at least one first nucleic acid of the two or more first nucleic acids.

In another embodiment, a process for magnetically detecting the presence of a target analyte in a sample. The process includes introducing a sample to an apparatus for magnetically detecting the presence of a target analyte in the sample. The process further includes determining the presence of the target analyte by determining release of at least one magnetic nanoparticle from at least one magnetic sensor of the apparatus at the time of the release of the at least one magnetic nanoparticle.

Implementations may include one or more of the following. The apparatus for magnetically detecting the presence of the target analyte may include: a plurality of magnetic nanoparticles; and a plurality of magnetic sensors disposed within a fluidic channel, each magnetic sensor of the plurality of magnetic sensors coupled, by a binding link, to at least one magnetic nanoparticle of the plurality of magnetic nanoparticles, the binding link adapted to be disrupted in the presence of a target analyte that breaks a covalent bond, a non-covalent bond, or both of the binding link to release the at least one magnetic nanoparticle from the magnetic sensor and indicate the presence of the target analyte. The apparatus for magnetically detecting the presence of the target analyte may further include processing circuitry configured to detect a signal from the magnetic sensor to indicate the absence of the at least one magnetic nanoparticle when the binding link is disrupted, thereby indicating the presence of the target analyte. The determining the presence of the target analyte may include: measuring a value of a characteristic of the magnetic sensor after introducing the sample; and determining a change in the value of the characteristic by comparing the measured value of the characteristic to a baseline value of the characteristic. Prior to introducing the sample, the process may further include introducing one or more materials to the sample, the one or more materials adapted to: increase a concentration of the target analyte in the sample from a first concentration of the target analyte to a second concentration of the target analyte, the binding link adapted to be disrupted by the second concentration of the target analyte; generate a species from the target analyte, the species different from the target analyte, the binding link adapted to be disrupted by the species; or combinations thereof. The target analyte may include a nucleic acid; and prior to introducing the sample, the process may further include introducing one or more materials to the sample, the one or more materials adapted to increase a concentration of the nucleic acid in the sample from a first concentration of the nucleic acid to a second concentration of the nucleic acid. The second concentration of the nucleic acid may cause activation of an enzyme present in the sample that breaks a covalent bond present in the binding link after introducing the sample to the apparatus. The second concentration of the nucleic acid may cause competitive displacement of a nucleic acid present in the binding link after introducing the sample to the apparatus. The second concentration of the nucleic acid may cause disassociation of a nucleic acid strand present in the binding link after introducing the sample to the apparatus.

### BRIEF DESCRIPTION OF THE DRAWINGS

So that the manner in which the above recited features of the present disclosure may be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 shows a general overview for magnetically detecting the presence and/or absence of an analyte of interest in a sample according to at least one embodiment of the present disclosure.

FIG. 2A shows a top view of an apparatus for magnetically detecting the presence and/or absence analyte of interest in a sample according to at least one embodiment of the present disclosure.

FIG. 2B shows a top view of an apparatus for magnetically detecting the presence and/or absence analyte of interest in a sample according to at least one embodiment of the present disclosure.

FIG. 2C shows optional processing/sensing circuitry utilized with apparatus described herein according to at least one embodiment of the present disclosure.

FIG. 3A shows a detection area of an apparatus for magnetically detecting the presence and/or absence of an analyte before running a sample comprising the analyte according to at least one embodiment of the present disclosure.

FIG. 3B shows a detection area of an apparatus for magnetically detecting the presence and/or absence of an analyte after running a sample according to at least one embodiment of the present disclosure.

FIG. 4 is a flowchart showing selected operations of a process for fabricating an apparatus for magnetically detecting the presence and/or absence of an analyte according to at least one embodiment of the present disclosure.

FIG. 5A shows selected operations of a process for magnetically detecting the presence and/or absence of an analyte according to at least one embodiment of the present disclosure.

FIG. 5B shows selected operations of a process for magnetically detecting the presence and/or absence of an analyte according to at least one embodiment of the present disclosure.

Figures included herein illustrate various embodiments of the disclosure. It is contemplated that elements and features of one embodiment may be beneficially incorporated in other embodiments without further recitation.

### DETAILED DESCRIPTION

Embodiments described herein generally relate to new apparatus and processes for magnetically detecting an analyte. The apparatus includes MNPs coupled to magnetic sensors by binding links. The chemistry of the binding link may be selected based on the analyte to be detected. In processes, for example, the analyte whose presence is to be detected is introduced to a fluidic channel of the apparatus. The analyte causes release or detachment of the MNP from the magnetic sensor by, for example, disrupting the binding link. Release of the MNP is accompanied by a signal output from the apparatus, e.g., as triggered by the sensed absence of the MNP by the corresponding magnetic sensor.

Disruption of the binding link refers to the analyte detaching, releasing, untethering, removing, or uncoupling the MNP from the magnetic sensor. The analyte may disrupt covalent bonds present in the binding link. Covalent bonds are chemical bonds involving the sharing of electrons to form electron pairs between atoms, such as C—N bonds, C—O bonds, C—C bonds, among other covalent bonds. For example, the analyte may disrupt a covalent bond of the binding link by breaking or cleaving a covalent bond present in the binding link. The breaking or cleaving of the covalent bond causes release of the MNP from the magnetic sensor.

Additionally, or alternatively, the analyte may disrupt non-covalent bonds present in the binding link. Non-covalent bonds, also referred to as non-covalent interactions, include: electrostatic interactions, such as ionic bonds, hydrogen bonds, or halogen bonds; van der Waals forces, such as dipole-dipole interactions, dipole-induced dipole interactions, or London dispersion forces (or induced dipole-induced dipole interactions); hydrophobic effects; and pi (π)-effects, such as π-π interactions, cation-π interactions, anion-π interactions, or polar-π interactions. For example, the analyte may alter ionic interactions, hydrogen bonding, dipole-dipole interactions, and/or London dispersion forces present in the binding link, causing release of the MNP from the magnetic sensor.

The analyte may disrupt both covalent bonds and non-covalent bonds present in the binding link. Whether the disruption involves covalent bonds, non-covalent bonds, or combinations thereof present in the binding link, the MNP becomes detached, untethered, or uncoupled from the magnetic sensor and the magnetic sensor will no longer detect the MNP and output a different signal than when the MNP was present. The absence of the MNP indicates the presence of the analyte to be detected within the sample.

Unlike conventional technologies, embodiments described herein may detect analytes by covalent or non-covalent disruption of a binding link. In addition, embodiments described herein may utilize a single MNP for detection of an analyte and do not require use of a plurality of particles. However, a plurality of MNPs may be utilized. In further contrast to state-of-the-art approaches, embodiments of the present disclosure may utilize in-line detection with magnetic sensors. For example, state-of-the-art approaches rely on washing beads (such as magnetic beads) away from a cleavage site and detecting the beads elsewhere.

In contrast, embodiments described herein provide real-time and in-line detection—the moment that a MNP detaches from the magnetic sensor, the movement of the MNP away from the sensor is detected. Even if the MNP sticks to a surface after detachment, the detachment is detected. Here, the inventor's approach is advantageous for at least the reason that the magnetic MNPs detach—which would be detected by embodiments herein—but then stick to a surface. This contrasts with conventional MNP-based sensing technologies whose sensing mechanisms rely on the MNPs binding to sensors, and such technologies would not detect such cases where the MNPs are sticking to a surface rather than binding to the sensors. In-line detection refers to the ability of embodiments described herein to detect release of at least one MNP at the time of release of at least one MNP from at least one magnetic sensor, due to a change in the magnetic field sensed by the magnetic sensor that is caused by movement of the MNP away from the magnetic sensor. That is, detection may occur the moment that a MNP detaches from the magnetic sensor. Such a mechanism provides a larger dynamic range relative to conventional technologies. In addition, embodiments described herein may be utilized to detect analytes of different concentrations by working on different time scales. For example, apparatus and processes described herein may be run until a “N” number of events have been detected rather than run for a time “X”. Such a mechanism automatically increases the detection sensitivity.

The use of headings is for purposes of convenience only and does not limit the scope of the present disclosure. Embodiments described herein may be combined with other embodiments. For example, features and elements of one described embodiment may be combined with other embodiments within the scope of the present disclosure.

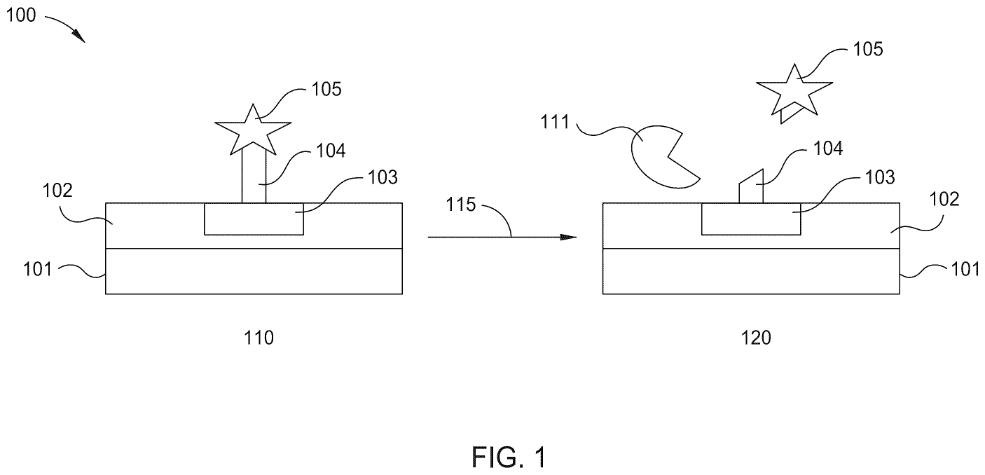

FIG. 1 shows a general overview 100 for magnetically detecting the presence and/or absence of an analyte of interest 111 (target analyte) in a sample according to at least one embodiment. Numerals 110 and 120 refer to embodiments prior to running a sample and after running the sample, respectively. The apparatus includes a wafer 101 and a fluidic channel 102 disposed over the wafer 101. A magnetic sensor 103, or plurality thereof, may be disposed on a surface of the fluidic channel and/or embedded within the fluidic channel 102. The magnetic sensor 103 may be a magnetoresistive sensor. Illustrative, but non-limiting, examples of magnetoresistive sensors may include magnetic tunnel junction (MTJ) sensors and giant magnetoresistance (GMR) sensors. MTJ sensors, relative to GMR sensors may provide higher magnetoresistance ratios and therefore higher sensitivity at low fields. Generally speaking, the magnetic sensor provides a signal output corresponding to an external magnetic field sensed by the sensor, which in that case may correlate to the presence or absence of an MNP in the vicinity of the sensor.

A binding link 104 couples MNP 105 to the magnetic sensor 103. The binding link 104 may include any suitable material that couples one or more MNPs 105 to the magnetic sensor 103 such as a molecule or plurality of molecules. The binding link 104 may be covalently coupled and/or non-covalently coupled to the magnetic sensor 103. The binding link 104 may be covalently coupled and/or non-covalently coupled to the MNP 105.

The binding link 104 is adapted to be disrupted in the presence of the analyte of interest 111. For example, the analyte of interest 111 may break a covalent bond, a non-covalent bond, or combinations thereof present in the binding link, thereby releasing or uncoupling the MNP from the magnetic sensor 103 and indicating the presence of the analyte of interest 111, via the sensor's signal output detecting the absence or departure movement of the MNP. The binding link may be adapted to be undisrupted in the absence of the analyte of interest 111, leaving the MNP coupled to the magnetic sensor 103 by the binding link 104 and indicating the absence of the analyte of interest 111.

At 120, after introducing a sample comprising the analyte of interest 111 into the fluidic channel 102 (operation 115), the analyte of interest 111 disrupts the binding link 104, causing release of the MNP 105 from the magnetic sensor 103. Here, a principle behind embodiments described herein is to specifically detach, untether, or otherwise uncouple the MNP 105 from the magnetic sensor 103 and the magnetic sensor 103 detects the change in the magnetic field generated during the detachment of the MNP 105. The field generated after the detachment has a value that is different from the value when the MNP 105 is attached to the sensor.

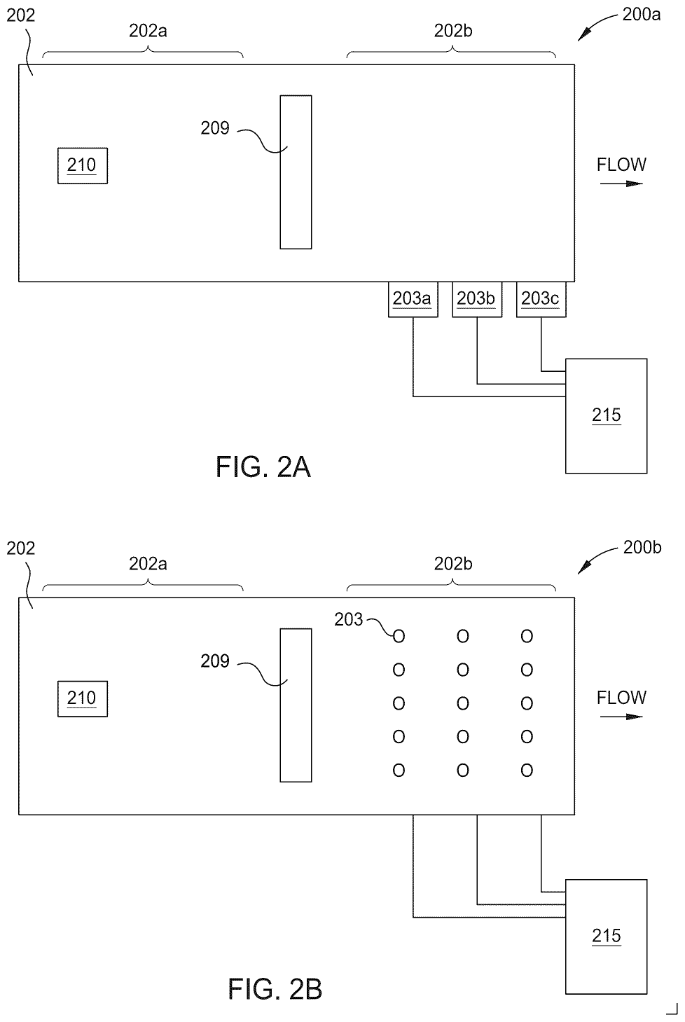

FIGS. 2A and 2B show top views of apparatus 200a and apparatus 200b, respectively, for magnetically detecting the presence and/or absence analyte of interest in a sample according to at least one embodiment. Apparatus 200a and apparatus 200b arc collectively referred to as apparatus 200. As described herein, the apparatus 200 may be an in-line detection apparatus, enabling, for example, detection of the release of at least one MNP at the time the MNP detaches from the magnetic sensor. The analyte of interest is the target analyte to be detected by the apparatus 200. A control analyte may also be present in the sample and may be run concurrently with the sample. The control analyte is distinct from the target analyte and may be utilized to ensure the sensor is functional during the test.

The apparatus 200 may be in the form of a microfluidic device or a lab-on-a-chip device. The apparatus 200 includes a fluidic channel 202. The fluidic channel 202 may have lateral dimensions from micrometers (μm) to millimeters, such as at least about 1 μm, such as from about 10 μm to about 2 mm, such as from about 50 μm to about 1 mm or from about 10 μm to about 1 mm. The fluidic channel 202 may be separated into at least two components—a sample introduction area 202a and a detection area 202b. The sample introduction area 202a may include an opening 210 through which the sample is introduced. The opening 210 is coupled to the fluidic channel 202. The opening 210 may range from a few micrometers in diameter to a few millimeters in diameter depending on the application.

The fluidic channel 202 may contain or otherwise be coupled to a one or more magnetic sensors 203, such as a plurality of magnetic sensors 203. The apparatus 200a shown in FIG. 2A shows three magnetic sensors 203a, 203b, and 203c (collectively, magnetic sensors 203). The one or more magnetic sensors 203 may be disposed within the fluidic channel 202. For example, the one or more magnetic sensors 203 may be disposed on a surface of the fluidic channel 202, embedded within the fluidic channel 202 or combinations thereof. As shown in FIG. 2A, the magnetic sensors 203 are shown on a side of the fluidic channel 202. Additionally, or alternatively, the magnetic sensors 203 (shown as “O”) may be located in the detection area 202b as shown in the apparatus 200b of FIG. 2B. With such designs, much of the sample fluid comes into contact with the magnetic sensors 203. As described above, a MNP may be attached to one or more of the magnetic sensors 203 via a binding link (not shown in FIGS. 2A and 2B).

The apparatus further includes one or more MNPs, such as a plurality of MNPs (e.g., MNPs 105, not shown in FIGS. 2A-2B). Each magnetic sensor of the one or more magnetic sensors 203 may be coupled, by a binding link, to at least one MNP of the one or more MNPs. Binding links 104 and MNPs 105 are shown in FIG. 1.

At least one magnetic sensor of the one or more magnetic sensors 203 may be coupled or attached to a binding link 104 that is disrupted by a particular analyte. For example, magnetic sensor 203a may be coupled to a first binding link that is disrupted by a first analyte. At least one magnetic sensor, for example, magnetic sensor 203b, of the one or more magnetic sensors 203 may be coupled or attached to a second binding link that is disrupted by a second analyte that is different from the first analyte. At least one magnetic sensor, e.g., magnetic sensor 203c, may be coupled or attached to a third binding link that is disrupted in the presence of a control analyte, the control analyte being different from the first analyte and second analyte. Here, the third binding link is adapted to be disrupted in the presence of the control analyte present in the sample to ensure the test was run. That is, the third binding link, in this example, is always disrupted by the control analyte.

As an illustrative, but non-limiting, example, and for the detection of different metals, the first binding link may be disrupted in the presence of copper (Cu). The second binding link may be disrupted in the presence of palladium (Pd) but may not be disrupted by Cu. The third binding link may be disrupted in the presence of a control analyte, for example, a non-metal. The non-metal may be added to the sample to ensure the test was run and/or that the sensors and the processor were fully functional.

The apparatus 200 may further include one or more processors 215 coupled to, e.g., the one or more magnetic sensors. The one or more processors 215 may be an external control circuit connected to apparatus 200.

The apparatus may optionally include an optional temperature control device 209. The optional temperature control device 209 may be coupled to the fluidic channel. Coupling of the fluidic channel 202 to the optional temperature control device 209 may take any suitable form. For example, the fluidic channel 202 may be coupled to the optional temperature control device 209 as shown in FIG. 2A, such as being embedded within the fluidic channel. As another example, the apparatus 200 may be placed on a hot plate (a temperature control device) and heated. Here, the hot plate may be located in a stand-alone machine outside of the example apparatus 100, and as such, the fluidic channel 202 is mechanically coupled to the optional temperature control device 209. As another example, the apparatus 200 may be placed under an infrared (IR) lamp (a temperature control device) and heated by the IR lamp. Here, the IR lamp may be located in a stand-alone machine outside of the apparatus 200, and as such, the fluidic channel 202 is optically coupled to the optional temperature control device 209. The optional temperature control device may be coupled to one or more processors 215.

The optional temperature control device 209 may be adapted to heat or cool the sample introduced into and flowing through the fluidic channel 202. The optional temperature control device 209 may be adapted to promote reaction between materials or components present the sample. Additionally, or alternatively, the optional temperature control device 209 may be adapted to promote disruption a binding link via an analyte. For example, the analyte may disrupt the binding link at elevated temperatures, but may not disrupt the binding link at room temperature (20° C. to 25° C.). As another example, the analyte may react with another material present in the sample, such as a reactant or an amplifier, at a specified temperature to form a reaction product. This reaction product may then disrupt the binding link.

Movement of the sample in the fluidic channel 202 from the opening 210 to the magnetic sensors 203 (in the direction of the arrow) may be controlled by, for example, capillary action, temperature, a pumping mechanism such as a piezoelectric pump, electrodes, and the like. Such elements may be placed along various parts of the fluidic channel 202. For example, the sample may be pumped by heating the sample and creating a bubble. The bubble may then act to push the sample towards the detection area. Additionally, or alternatively, the apparatus 200 may include pumping elements to pump, or draw, the sample to the magnetic sensors 203.

Pumping may be achieved by use of a simple syringe operated manually, or with a micro-pump (for example, a small micro-machined pump of dimension comparable to the analysis unit), a membrane pump, or with any other suitable type of pumping system capable of producing sufficient suction to pump or draw the sample in apparatus 200. The pumping elements may be located at various positions along the fluidic channel 202, such as in the sample introduction area 202a, the detection area 202b, or both. For example, pumping elements may be placed between the sample introduction area 202a and the detection area 202b to pump the sample from the opening 210 to the detection area 202b. Apparatus 200 may be powered by any suitable means including, but not limited to, batteries, AC power supply, DC power supply, and the like.

During operation, the sample is introduced to the fluidic channel 202 through the opening 210 and flows from the sample introduction area 202a to the detection area 202b. As described herein, the magnetic sensors 203 are coupled to MNPs (for example, MNP 105) via binding links (for example, binding link 104). An analyte present in a fluid sample, for example, analyte of interest 111, interacts with, or disrupts, the binding link, resulting in detachment or uncoupling of the MNP from the magnetic sensor 203 and detection of the analyte of interest 111.

The apparatus may further include processing/sensing circuitry configured to detect a signal from the magnetic sensor to indicate the absence of the MNP when the binding link is disrupted, thereby indicating the presence of the target analyte. For example, and as shown in FIG. 2C, the apparatus 200 may further include processing/sensing circuitry 230 coupled to one or more of the magnetic sensors 203 via line 235. The processing/sensing circuitry 230 may be coupled to the one or more processors 215. The processing/sensing circuitry senses and detects the disruption of the binding link. The processing/sensing circuitry 230 may be adapted to determine a characteristic or a change in characteristic for the magnetic sensors 203, wherein the characteristic or change in characteristic is influenced by the presence or absence of the MNP. In one example, in magnetoresistive sensors such as tunnel magnetoresistance (TMR), an orientation change of the magnetic layers of the sensors caused by changes in the sensed external field drives a electrical resistance change, which may be converted to a detection signal. However, those skilled in the art would recognize various other characteristics of the sensors indicative of the MNP's absence/presence that may be used. The presence of the MNP means that the MNP is coupled to the magnetic sensor by the binding link and there is no analyte, or an insufficient amount of analyte, is present. The absence of the MNP means that the MNP is no longer coupled to the magnetic sensor by the binding link. The presence and absence of the MNP have different signal outputs corresponding to different characteristics, or values thereof, of the magnetic sensor.

As further described herein, magnetically detecting an analyte may involve determining the presence and/or absence of the analyte. Determining the presence and/or absence of the analyte may include detecting, monitoring, and/or measuring a change in a characteristic between the state of the magnetic sensors 203 prior to introduction of the sample and the state of the magnetic sensors 203 at some time after introduction of the sample. The characteristic, or value thereof, may be determined using the processing/sensing circuitry 230.

In some embodiments, in operation, the processing/sensing circuitry 230 applies, for example, a current and/or a voltage to the line 235 to detect, monitor, measure, or otherwise determine a characteristic of at least one of the magnetic sensors 203, where the characteristic indicates the presence of the MNP (corresponding to the absence of, or insufficient amount of, the analyte) and/or an absence of MNP (corresponding to the presence of the analyte). Suitable characteristics, or changes thereof, that may be detected, monitored, measured, or otherwise determined may include resistance, magnetic field, current, voltage, voltage drop, magnetic noise, noise level, jitter, noise variance, a change thereof, or combinations thereof. For example, determining the presence or absence of the analyte may be performed by applying a current and measuring the characteristic or value thereof. Additionally, or alternatively, determining the presence or absence of the analyte may be performed by applying a voltage and measuring the characteristic. By taking the measurements, the presence and/or absence of the analyte in the sample may be monitored in real-time.

Any suitable processing/sensing circuitry may be utilized for processing/sensing circuitry 230 such as processing/sensing circuitry described in, for example, U.S. patent application Ser. No. 16/659,383, titled “Magnetoresistive Sensor Array for Molecule Detection and Related Detection Schemes,” filed Oct. 21, 2019, which is incorporated herein by reference in its entirety. For example, the processing/sensing circuitry 230 may include a bias element coupled to at least one magnetic sensor and adapted to generate a bias across the at least one magnetic sensor. The processing/sensing circuitry 230 may further include a first low pass filter and amplifier combination coupled to the at least one magnetic sensor to filter and amplify a signal from the at least one magnetic sensor. The processing/sensing circuitry 230 may further include a reference oscillator configured to generate a reference signal having a particular frequency chosen to maximize a change in the signal at the particular frequency when an MNP is detected by the corresponding magnetic sensor. The processing/sensing circuitry 230 may further include a mixer coupled to the reference oscillator and an output of the first low pass filter and amplifier combination, wherein the mixer is adapted to mix an output signal from the first low pass filter and amplifier combination with the reference signal. The processing/sensing circuitry 230 may further include a second low pass filter and amplifier combination coupled to the mixer. The processing/sensing circuitry 230 may further include an envelope detector adapted to receive an output signal from the second low pass filter and amplifier combination and provide a signal for detection.

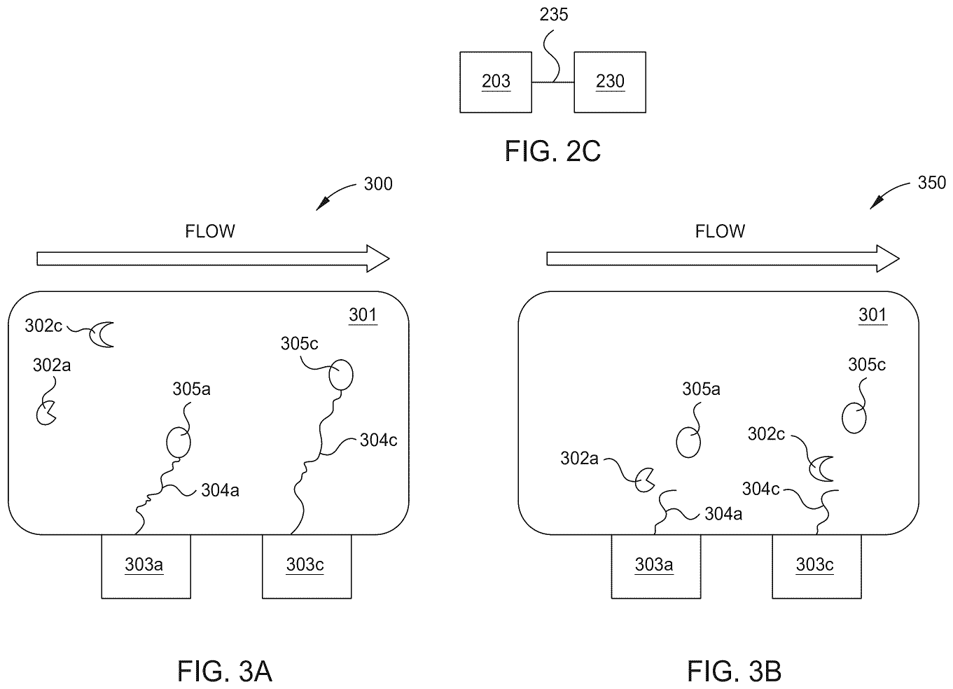

FIGS. 3A and 3B show a detection area of an apparatus for magnetically detecting the presence and/or absence of an analyte before (300) and after (350) running a sample, respectively. The detection areas 300, 350 may correspond to detection area 202b of FIGS. 2A and 2B. 302a is a target analyte to be detected and 302c is a control analyte utilized to ensure the test was run and/or the sensors and the processor were functional.

MNP 305a is coupled to magnetic sensor 303a by binding link 304a. The magnetic sensor 303a is adapted to detect the presence of MNP 305a (corresponding to the absence of, or insufficient amount of, the target analyte 302a) and/or an absence of MNP (corresponding to the presence of the target analyte 302a). Binding link 304a is adapted to be disrupted by the target analyte 302a. That is, binding link 304a is selective or specific for the target analyte 302a.

MNP 305c is coupled to magnetic sensor 303c by binding link 304c. The magnetic sensor 303c is adapted to detect the presence of MNP 305c (corresponding to the absence of, or insufficient amount of, the control analyte 302c) and/or an absence of MNP (corresponding to the presence of the control analyte 302c). Binding link 304c is adapted to be disrupted by control analyte 302c; that is, binding link 304c is selective or specific for the control analyte 302c. The control analyte 302c is different from the target analyte 302a.

As the analytes, for example, target analyte 302a and control analyte 302c, flow into the detection area, the analytes may interact with and disrupt the corresponding binding links, for example, binding link 304a and binding link 304c, respectively. Upon disruption of the corresponding binding link, the corresponding MNP (for example, MNP 305a or MNP 305c, respectively) becomes detached, untethered, or uncoupled from the corresponding magnetic sensor and a determination of the analyte(s) present or absent may be made.

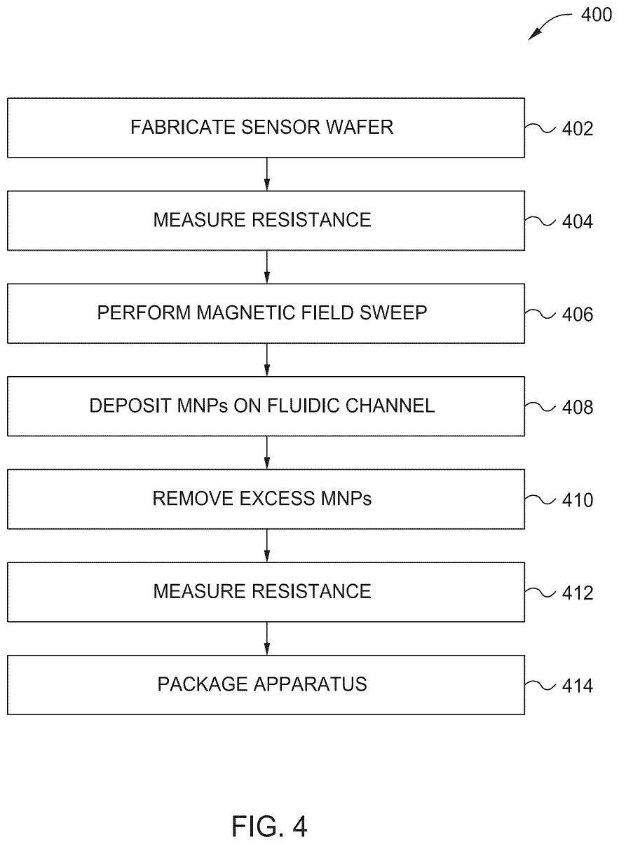

Embodiments described herein also generally relate to processes for fabricating apparatus described herein. FIG. 4 is a flowchart showing selected operations of a process 400 for fabricating an apparatus (a sensor device) for magnetically detecting the presence and/or absence of an analyte according to at least one embodiment. Although the fabrication process involves testing the apparatus with resistance measurements for quality control, other characteristics or changes thereof may be measured to test the apparatus such as a magnetic field, a current, a voltage, a voltage drop, a magnetic noise, a noise level, jitter, a noise variance, a change thereof, or combinations thereof.

The process 400 begins at operation 402 with fabricating a sensor wafer. The process of operation 402 may include fabricating a wafer 101 and a fluidic channel 102 disposed over the wafer 101. The wafer 101 and fluidic channel 102 may be formed by any suitable method. The wafer 101 may be a silicon wafer. The fluidic channel 102 may be formed using polymer chemistry.

The polymers may be formed from monomers such as vinyls, acrylates, methacrylates, urethanes, siloxanes, epoxy resins, thiol-enes, among others. Forming the fluidic channel 102 may involve creating a mold which may be replicated by mechanical means (for example, microcutting or ultrasonic machining), energy-assisted means (for example, electrodischarge, micro-electrochemical, laser ablation, electron beam, or focused ion beam), traditional micro-electromechanical systems (MEMS), among others.

For example, a photoresist may be spin coated onto the wafer, exposed to UV light, and annealed for patterning. Polydimethylsiloxane may be deposited onto the wafer and plasma treated. Magnetic sensors 103 may then be integrated into the apparatus by any suitable method, and inlets and outlets may be formed in the apparatus.

At operation 404, resistance R0 values of the magnetic sensors 103 of the sensor wafer are then measured. R0 refers to the resistance value of the sensor when no MNP is coupled to it. During operation 402, defective sensors may be identified. A magnetic field sweep is then performed at operation 406 to, e.g., test the sensitivity of a magnetic sensor 103 of the sensor wafer.

MNPs 105 may then be deposited on the magnetic sensors 103 at operation 408. The process of operation 408 may include coupling a binding link 104 to the magnetic sensor 103 and coupling an MNP 105 to the binding link. This is unlike typical magnetic sensing devices which are not made with binding links and MNPs at time of manufacture.

The magnetic sensor-binding link-MNP system may be held together by covalent or non-covalent interactions. For example, the binding link 104 may be coupled to the magnetic sensor 103 by first introducing reactive functional groups (for example, epoxides) to a surface of the fluidic channel 102. The binding link 104 may be bonded with the surface of the fluidic channel by reacting with the reactive functional groups on the surface of the fluidic channel 102. For example, the binding link 104 may include reactive functional groups (for example, amines) that react with the epoxide functional groups on the surface of the fluidic channel 102. MNPs 105 may then be covalently attached to the binding link 104. For example, the MNPs 105 may include reactive functional groups, such as carboxyl groups, that chemically react with functional groups present on the binding link 104.

As another example, DNA as a binding link 104 may be immobilized on a magnetic sensor 103 by forming a polymethyl methacrylate (PMMA) surface on the magnetic sensor 103 and photocrosslinking the DNA to the PMMA surface. Functional groups present on MNPs 105 may then be used to form couple the MNPs 105 and the DNA binding link using standard chemistry methods.

As another example, polypeptides (e.g., proteins) may be immobilized on a magnetic sensor 103 by forming a polydimethylsiloxane (PDMS) surface on the magnetic sensor 103, followed by passive adsorption of 1-ethyl-3-(3-dimethylaminopropyl) carbodiimide hydrochloride, followed by cross-linking with a polypeptide. Reactive functional groups present on MNPs 105 may then be used to couple the MNP with the polypeptide binding link using standard chemistry methods.

As another example, lipids, phospholipids, or lipid mimics may be immobilized on a magnetic sensor 103 by first coating the surface of the magnetic sensor 103 with saturated or unsaturated fatty acids. The lipids, phospholipids, or lipid mimics may then be deposited on the saturated or unsaturated fatty acids according to known methods. Then MNPs 105, coated with saturated or unsaturated fatty acids, may then be deposited on the lipids, phospholipids, or lipid mimics. Magnetoliposomes may be utilized. Magnetoliposomes are MNPs coated with saturated or unsaturated fatty acids with a number of carbon atoms ranging between, for example, 4 and 30 carbon atoms, for example dodecanoic acid or (9Z)-octadec-9-enoic acid. These magnetoliposomes may be deposited onto the fatty-acid coated surface of the magnetic sensor 103. Additionally, or alternatively, magnetoliposomes may be incorporated into liposome vesicles, and the liposome vesicles deposited on the surface of the magnetic sensor 103.

Additionally, or alternatively, the MNP 105 may be coupled to the magnetic sensor 103 without forming covalent bonds. Here for example, the binding link 104 may include a non-polar molecule such as grease or wax. The non-polar molecule may be deposited onto the fluidic channel 102, and then MNPs 105 may be deposited on the non-polar molecule. The non-polar molecule as a binding link 104 serves to couple, or attach, the MNPs 105 to the magnetic sensors 103. This sensor-non-polar molecule-MNP system may be used to test analytes such as solvents, surfactants, etc.

The process 400 may further include removing excess MNPs from the sensor wafer at operation 410. Removing excess MNPs may be performed by a washing operation. The process 400 may further include measuring the resistance (R1) of the sensor wafer at operation 412. Operation 412 may include recording resistance R1 values of the magnetic sensors 103. If R1≈R0, the magnetic sensor 103 is not coupled to the MNP 105. If the difference of R1 and R0 is of the expected sign (+ or −) and within a range of expected magnitudes, the magnetic sensor 103 is coupled to the MNP 105. At this stage, the sensor device may then be packaged at operation 414 to form apparatus described herein, and an optional final resistance measurement may be performed on the apparatus.

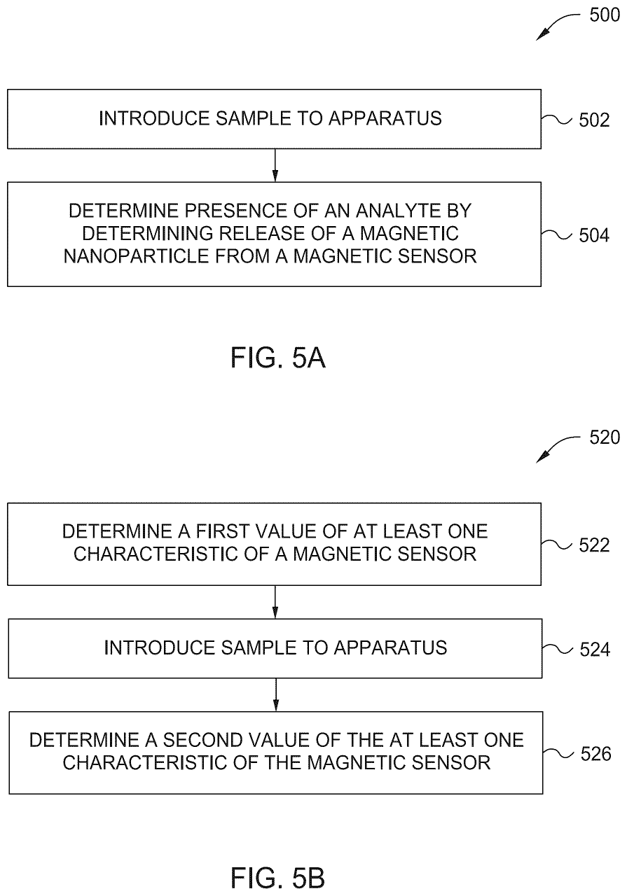

Embodiments described herein also generally relate to processes for magnetically detecting the presence and/or absence of an analyte. FIG. 5A shows selected operations of a process 500 for magnetically detecting the presence and/or absence of an analyte according to at least one embodiment.

The process 500 begins with introducing a sample to an apparatus described herein at operation 502. The apparatus may be apparatus 200a or apparatus 200b. The introducing process of operation 502 may include pipetting a sample into an opening of the apparatus, inserting a swab into an opening of the apparatus, or other suitable methods of introducing the sample. For example, a sample may be introduced into opening 210 of apparatus 200a.

The process 500 may further include determining the presence and/or absence of a target analyte in the sample at operation 504. Determining the presence and/or absence of the target analyte at operation 504 may involve determining release of at least one MNP from at least one sensor. Determining the presence and/or absence of the target analyte may involve detecting, monitoring, measuring, or otherwise determining a characteristic, or value thereof, of at least one of the magnetic sensors, where the characteristic indicates the presence of the MNP (corresponding to the absence of, or insufficient amount of, the analyte) and/or an absence of MNP (corresponding to the presence of the analyte). Suitable characteristics, or changes thereof, that may be detected, monitored, measured, or otherwise determined may include resistance, magnetic field, current, voltage, voltage drop, magnetic noise, noise level, jitter, noise variance, a change thereof, or combinations thereof.

Additionally, or alternatively, the presence of the target analyte may be determined by measuring a value of the characteristic of the magnetic sensor after introducing the sample and then determining a change in the value of the characteristic by comparing the measured value of the characteristic to the baseline value of the characteristic. The baseline value may be the value measured prior to the process and/or may be a value measured from the control sensor during the process.

Additionally, or alternatively, the presence of the target analyte may be determined by measuring a plurality of values of a characteristic of the magnetic sensor, and fitting a curve to these plurality values to determine a derived value. The derived value may then be compared to a baseline value of the characteristic of the magnetic sensor.

Additionally, or alternatively, the presence of the target analyte may be determined by measuring a value of a characteristic of the magnetic sensor, and determining measurement parameters (conditions under which the process is performed, e.g., temperature, wait time—a period of time after the sample is introduced to the apparatus, —among other conditions). The value of the characteristic of the magnetic sensor at the measurement parameter may then be compared to a baseline characteristic of the magnetic sensor at the measurement parameter.

Here, for example, the presence of the target analyte 302a in the sample disrupts the binding link 304a that couples the MNP 305a to the magnetic sensor 303a, causing release, untethering, or uncoupling of the MNP 305a from the magnetic sensor 303a. As a result, the resistance at the magnetic sensor 303a will change. That is, the presence of a target analyte may be determined by release of the MNP 305a from the magnetic sensor 303a. When the MNP 305a is not released from the magnetic sensor 303a, the target analyte 302a may not be present in the sample or may not be present in the sample at sufficient concentrations. Accordingly, the lack of a change in resistance at the magnetic sensor 303a may indicate that the target analyte 302a is not present in the sample or is not present in sufficient concentrations to disrupt the binding link 304a.

As described herein, the apparatus may include a control sensor (for example, magnetic sensor 303c). The control sensor is coupled to a MNP via a binding link, for example, MNP 305c and binding link 304c. Accordingly, and in some embodiments, process 500 may include introducing a control analyte, e.g., control analyte 302c, to the sample. The process 500 may further include determining the presence of the control analyte in the sample by monitoring a change in resistance of the control sensor. Here, for example, the presence of the control analyte 302c in the sample disrupts the binding link 304c that couples the MNP 305c to the magnetic sensor 303c, causing release, untethering, or uncoupling of the MNP 305c from the magnetic sensor 303c. As a result, the resistance at the magnetic sensor 303c will change. That is, the presence of a control analyte 302c may be determined by release of the MNP 305c from the magnetic sensor 303c. When the MNP 305c is not released from the magnetic sensor 303c, the control analyte 302c may not be present in the sample or may not be present in the sample at sufficient concentrations. Accordingly, the lack of a change in resistance at the magnetic sensor 303c may indicate that the control analyte 302c is not present or is not present in sufficient concentrations to disrupt the binding link 304c.

Unlike conventional methods, the process 500 enables in-line and/or real-time detection. The presence of the target analyte and/or control analyte may be determined by release of at least one MNP from at least one magnetic sensor at the time of the release of the at least one MNP from the at least one magnetic sensor. That is, detection may occur the moment that a MNP detaches from the magnetic sensor.

FIG. 5B shows selected operations of a process 520 for magnetically detecting the presence and/or absence of analyte according to at least one embodiment. Embodiments of process 520 may be combined with embodiments of process 500.

The process 520 begins with determining a first value of at least one characteristic of a magnetic sensor at operation 522. The determining the first value of the at least one characteristic may include detecting, monitoring, or measuring the characteristic via, e.g., processing/sensing circuitry 230. Suitable characteristics that may be detected, measured, monitored, or otherwise determined may include resistance, magnetic field, current, voltage, voltage drop, magnetic noise, noise level, jitter, noise variance, a change thereof, or combinations thereof. For example, the processing/sensing circuitry 230 may apply, for example, a current and/or a voltage to the line 235 to detect, monitor, measure or otherwise determine a characteristic of at least one of the magnetic sensors 203, where the characteristic indicates a presence or an absence of a MNP.

The process 520 further includes introducing a sample to an apparatus for magnetically detecting an analyte at operation 524. Suitable apparatus for magnetically detecting an analyte are described herein such as apparatus 200a or apparatus 200b.

The sample may optionally include one or more optional materials such as a control analyte, a reactant, a reagent, an enzyme, a primer, an amplifier, or combinations thereof, among others. The one or more optional materials may be introduced to the sample prior to the introducing the sample to the apparatus or may be introduced to the apparatus separately from the sample. The introducing process of operation 524 may include pipetting a sample into an opening of the apparatus, inserting a swab into an opening of the apparatus, or other suitable methods for introducing the sample. For example, a sample may be introduced into opening 210 of apparatus 200a.

The process 520 further includes determining a second value of the at least one characteristic of the magnetic sensor at operation 526. The process of operation 526 may be performed in the same or similar manner as described above for operation 522. For example, the processing/sensing circuitry 230 may apply, for example, a current and/or a voltage to the line 235 to detect, monitor, measure or otherwise determine a characteristic of at least one of the magnetic sensors 203, where the characteristic indicates a presence or an absence of a MNP. Comparison of the first value of the characteristic to the second value of the characteristic allows determination of whether the MNP released from the magnetic sensor.

In operation, and in some embodiments, a first resistance value (R^{A}) of a magnetic sensor may be determined at operation 522. Additionally, or alternatively, the R^{A} may be a known baseline value measured before performing process 520. A sample may be introduced to the apparatus at operation 524. A second resistance value (R^{B}) of the magnetic sensor may then be determined at operation 526. Release, detachment, or uncoupling of the MNP from the magnetic sensor may be indicated by R^{B}≈R0, indicating that the binding link is disrupted. Additionally, or alternatively, release of the MNP from the magnetic sensor may be indicated when R^{A} and R^{B} are sufficiently different values.

Unlike conventional methods, the process 520 enables in-line and/or real-time detection. The presence of the target analyte and/or control analyte may be determined by release of at least one MNP from at least one magnetic sensor at the time of the release of the at least one MNP from the at least one magnetic sensor. That is, detection may occur the moment that a MNP detaches from the magnetic sensor.

As described above, the binding link may be adapted to be disrupted in the presence of an analyte, and may be adapted to be undisrupted in the absence of the analyte. In some embodiments, which may be combined with other embodiments, the binding link 304a may be adapted to be disrupted in the presence of a composition. As used herein, a “composition” may include component(s) of the composition, reaction product(s) of two or more components of the composition, a remainder balance of remaining starting component(s), or combinations thereof.

Such compositions may be formed by optionally introducing one or more materials to the sample. The one or more materials that may be optionally added to the sample may include a reactant, a reagent, an enzyme, a primer, an amplifier, an inhibitor, or combinations thereof, among other optional materials.

The composition that disrupts the binding link 304a may include a reaction product of a target analyte reacting with a reactant. In this example, embodiments described herein may be used for indirect detection of a target analyte by detecting reaction product A′ according to Scheme 1.

$$A + B \to {A}^{′} \qquad Scheme 1$$

Here, for example, the target analyte serves as a precursor target analyte. Upon reaction of the precursor target analyte A with the reactant B, a reaction product A′ is formed. The reaction product A′ interacts and disrupts the binding link 304a indicating the presence of the precursor target analyte A. For this indirect detection, the process may further include introducing one or more materials to the sample, at least one of the one or more materials is adapted to react with an analyte present in the sample and form a reaction product that disrupts the binding link.

Processes described herein may be utilized to detect target analytes present in a sample at low concentrations. In some embodiments, which may be combined with other embodiments, detecting a sample at low concentrations may be performed by chemical amplification. Chemical amplification may involve a chemical reaction that increase the concentration of the analyte from a first concentration of the analyte to a second (and higher) concentration of the analyte. Here, the binding link may be adapted to be disrupted in the presence of the second concentration of the analyte and may be adapted to be undisrupted in the presence of the first concentration of the analyte. Additionally, or alternatively, chemical amplification may involve a chemical reaction that generates a new species from the analyte, where the binding link may be adapted to be disrupted in the presence of the new species and may be undisrupted in the presence of the analyte.

An embodiment of chemical amplification is shown in Scheme 2.

$$A + D ⇌ AD AD + C ⇌ AD + {C}^{′} \qquad Scheme 2$$

wherein: A is the target analyte; D is a reactant that reacts with A; AD is a reaction product; C is an amplifier and C′ disrupts the binding link.

According to Scheme 2, the target analyte A reacts with a reactant D to form a reaction product AD. The reaction product AD reacts with an amplifier to form a reaction product C′ which disrupts the binding link. AD may serve as a catalyst. By utilizing high concentrations of reactant D and amplifier C, high concentrations of C′ are produced from low concentrations of target analyte A. Further and other embodiments of chemical amplification are described below.

As described herein, apparatus and processes of the present disclosure may be utilized to detect analytes of different concentrations. For example, processes described herein may be performed until a specified number of MNPs have been released from their corresponding magnetic sensors. Here, the specified number of MNPs released from their corresponding magnetic sensors corresponds to a N number of events. The process may be run N until an N number of events has occurred. For example, when two different threshold concentrations of an analyte are desired to be determined, determining the first (lower) concentration may involve one or more events-one or more MNPs being released, detached, or uncoupled from the their corresponding magnetic sensors. Determining the second (higher) concentration, may involve two or more events-two or more MNPs being released, detached, or uncoupled from their corresponding magnetic sensors.

In some embodiments, which may be combined with other embodiments, processes may include performing a baseline correction. Baseline correction may permit removal of variations in sensitivity, temperature, chemicals, electronics, environmental effects (external magnetic field), contamination (e.g., due to the MNPs), etc. The baseline correction may utilize a ratio of the target analyte detected by release of the MNP 305a from the magnetic sensor 303a versus the control analyte detected by release of the MNP 305c from the magnetic sensor 303c. Other methods for baseline correction are contemplated.

Embodiments described herein may be used to determine the presence and/or absence of any suitable analyte within a sample. These analytes may be minor constituents of the sample and may therefore be challenging to detect by conventional technologies. Moreover, any suitable type of molecule that may disrupt a binding link—by, e.g., breaking a covalent bond, a non-covalent bond, or combinations thereof—may be detected utilizing embodiments of the present disclosure. Thus, embodiments of the present disclosure may extend to molecules such as biological molecules, organic molecules, inorganic molecules, minerals, and transition metals, among others.

In addition, embodiments described herein may be used as a high-throughput method for testing how various analytes interact with various substances. For example, molecules that are not known to disrupt deoxyribonucleic acid (DNA) may be tested for their ability to disrupt DNA using apparatus and processes described herein.

### Example Analytes and Magnetic Sensor—Binding Link—MNP

Detection of Analytes that Disrupt Nucleic Acids

Embodiments described herein may be used to detect analytes, present in a sample, that disrupt a nucleic acid. For this example, the binding link may include a nucleic acid, such as DNA. The analyte to be detected may include an enzyme, a mutagenic chemical, or combinations thereof. Enzymes and mutagenic chemicals may disrupt the chemical structure of DNA by breaking chemical bonds present in the DNA. For example, many mutagenic chemicals are electrophiles that form covalent reaction products with nucleophilic sites within DNA. Similarly, enzymes have reactive functional groups that react with specific reactive sites of DNA, for example, by hydrolysis. Additionally, or alternatively, the analyte to be detected may disrupt a DNA by breaking non-covalent bonds, such as hydrogen bonds, present in DNA. In action, for example, the analyte may disrupt the nucleic acid-containing binding link by breaking covalent bonds and/or non-covalent bonds present in the nucleic acid of the binding link, thereby releasing the MNP from the magnetic sensor.

Beyond enzymes, various analytes that disrupt nucleic acids that may be detected using embodiments described herein may include, but are not limited to, deaminating agents, polycyclic aromatic hydrocarbons, alkylating agents such as vinyl chloride, nitrosamines such as ethylnitrosourea, aromatic amines such as proflavine, aromatic amides such as 2-acetylaminofluorene, various alkaloids, bromine-containing molecules, and intercalating agents, among others.

### Detection of Surfactants

Embodiments described herein may be used to detect molecules that disrupt surface tension between two phases, e.g., surfactants, present in a sample. Surfactants are molecules that decrease surface tension between two phases.

In this example, the analyte may include a surfactant. Any suitable binding link that a surfactant disrupts may be used for testing the analyte. For example, the binding link may include nonpolar molecules such as oil, grease, wax, or combinations thereof, among others. In action, for example, the surfactant may disrupt the binding link by breaking non-covalent bonds of the binding link, thereby releasing the MNP from the magnetic sensor. For example, the surfactant may form micelles around oil soluble binding links (nonpolar molecules), facilitating release of the MNP from the magnetic sensor.

Surfactants that may be detected with embodiments described herein may include anionic surfactants, cationic surfactants, zwitterionic surfactants, non-ionic surfactants. Anionic surfactants are surfactants that contain an anionic functional group at their head such as a sulfonate group, sulfate group, carboxylate group, or phosphate group. Cationic surfactants are surfactants that contain a cationic functional group at their head such as pH-dependent amines such as primary, secondary, or tertiary amines, as well as quaternary ammoniums. Zwitterionic surfactants are surfactants that contain both a positive and negative charge. Zwitterionic surfactants include amine oxides. Other zwitterionic surfactants include both a cationic functional group and anionic functional group, for example, those that include an amine or ammonium group as the cationic functional group and a carboxylate, sulfonate, or phosphate as the anionic functional group, among others. Non-ionic surfactants include surfactants that are neutral. Nonionic surfactants typically include oxygen containing hydrophilic moieties bonded to hydrophobic structures. Nonionic surfactants may include ethoxylates, fatty alcohol ethoxylates, alkylphenol ethoxylates, fatty acid ethoxylates, ethoxylated fatty esters, ethoxylated fatty oils, ethoxylated amines, ethoxylated fatty acid amines, terminally blocked ethoxylates (e.g., poloxamers), fatty acid esters of polyhydroxy compounds, fatty acid esters of glycerol, fatty acid esters of sorbitol, fatty acid esters of sucrose, alkyl polyglucosides, among others.

### Detection of Low Polarity Solvents

Embodiments described herein may be used to detect low polarity solvents present in a sample. In this example, the analyte includes a low polarity solvent (relative to water), such as phenol, and the binding link includes any suitable material disrupted by the low polarity solvent. Low polarity solvents may induce conformational changes in streptavidin-biotin. Accordingly, and in some embodiments, the binding link includes streptavidin-biotin.

The bond between biotin and streptavidin is a non-covalent bond. In action, for example, a low polarity solvent such as phenol may disrupt non-covalent bond(s) present in the streptavidin-biotin binding link, releasing the MNP from the magnetic sensor.

### Detection of Enzymes

Embodiments described herein may be utilized to detect an enzyme present in a sample. For this example, the analyte may include any suitable enzyme and the binding link may include any molecule that is disrupted by the enzyme. For example, certain enzymes, such as restriction endonucleases, which may be tested with binding links that include a nucleic acid, as restriction endonucleases are known to break covalent bonds in nucleic acids such as DNA. Other examples of enzymes may include serine proteases which may be tested with binding links that include peptides. Serine proteases are known to break covalent bonds present in peptides. In action, for example, the enzyme to be detected may disrupt covalent bond(s) present in the binding link (e.g., a nucleic acid, a peptide, among others), releasing the MNP from the magnetic sensor.

In some embodiments, which may be combined with other embodiments, the sample introduced into the apparatus may optionally include one or more materials (e.g., a reactant, reagent, etc.) that the enzyme binds with to form a reaction product and the reaction product may disrupt the binding link. Additionally, or alternatively, the sample introduced into the apparatus may optionally include one or more materials (e.g., a reactant, reagent, etc.) that the enzyme binds with to begin a cascade of events that result in a reaction product, and the reaction product may disrupt the binding link. As such, indirect detection of enzymes is enabled by embodiments of the present disclosure. Indirect detection may be a form of chemical amplification.

In some embodiments, which may be combined with other embodiments, the analyte introduced into the apparatus may bind to the enzyme forming a reaction product without or with lowered enzymatic activity. Additionally, or alternatively, the sample introduced into the apparatus may optionally include one or more materials (e.g., a reactant, reagent, etc.) that the analyte binds with to begin a cascade of events that result in the inactivation of the enzyme, where the inactivated enzyme does not disrupt the binding link or does so at a reduced rate compared to the non-inactivated enzyme. As such, indirect detection of analytes is enabled by embodiments of the present disclosure. Indirect detection may be a form of chemical amplification.

### Detection of Solvents

Embodiments described herein may be utilized to detect solvent(s) present in a sample. For this example, the solvent may be any suitable solvent such as polar or non-polar, protic or aprotic, ionic or non-ionic. Depending on the type of solvent that is desired to be detected, the binding link may alter.

For example, polystyrene may be used as a binding link. Polystyrene is soluble in ethyl acetate, dichloromethane (DCM), dimethylformamide (DMF), dimethylsulfoxide (DMSO), and toluene, but is insoluble in water, low molecular weight alcohols, diethyl ether, and hexanes. Therefore, polystyrene may be used as a binding link to detect ethyl acetate, DCM, DMF, DMSO, and toluene. In action, for example, such analytes may disrupt the binding link by solubilizing the polystyrene—breaking of non-covalent bonds such as intermolecular forces of the polystyrene—and release the MNP from the magnetic sensor. For example, upon disrupting the polystyrene by solubilizing the polystyrene (breaking of non-covalent bonds), the MNP is released from the magnetic sensor.

Ionic solvents, e.g., ionic liquids, may also be tested as analytes. For this example, the binding link may include a salt. Salts include ionic bonds which are non-covalent bonds. In action, for example, the ionic liquid analyte may disrupt the salt binding link by breaking non-covalent bonds of the salt, thereby releasing the MNP from the magnetic sensor.

Additionally, or alternatively, certain solvents alter conformations of biological structures, such as proteins. The conformation of the protein may be determined by non-covalent bonds present in the protein. For this example, a solvent having a polarity different than water may be detected. In action, for example, a solvent having a different polarity than water may disrupt non-covalent bond(s) present in the streptavidin-biotin binding link, releasing the MNP from the magnetic sensor.

### Detection of Transition Metals

Embodiments described herein may be utilized to detect transition metal(s) or transition metal-containing compounds present in a sample. Transition metals may include one or more elements from Group 3 to Group 12 of the periodic table of the elements such as scandium (Sc), yttrium (Y), titanium (Ti), zirconium (Zr), hafnium (Hf), rutherfordium (Rf), vanadium (V), niobium (Nb), tantalum (Ta), dubnium (Db), chromium (Cr), molybdenum (Mo), tungsten (W), seaborgium (Sg), manganese (Mn), technetium (Tc), rhenium (Re), bohrium (Bh), iron (Fe), ruthenium (Ru), osmium (Os), hassium (Hs), cobalt (Co), rhodium (Rh), iridium (Ir), meitnerium (Mt), nickel (Ni), palladium (Pd), platinum (Pt), darmstadtium (Ds), copper (Cu), silver (Ag), gold (Au), roentgenium (Rg), zinc (Zn), cadmium (Cd), mercury (Hg), copernicum (Cn), or combinations thereof.

Various transition metals, and compounds containing transition metals, may break covalent bonds, such as C—N bonds (such as a C—N bond of a carbamate moiety), C—O bonds (such as C—O bonds of an ether moiety), C—N bonds (such as a C—N bond of an amide moiety), or combinations thereof. As an example, transition metals and/or transition metal-containing compounds may be used for disrupting one or more of the following covalent bonds (bond cleavage) present in a binding link:

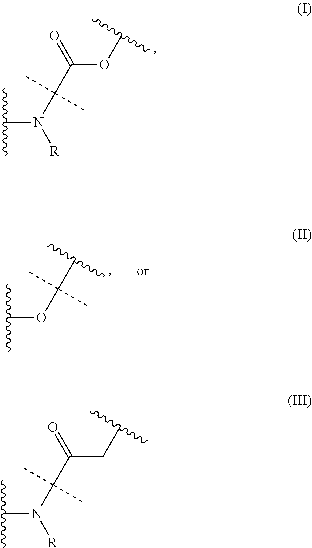

wherein:
the dashed lines in formula (I), formula (II), and formula (III) indicates the covalent bond broken by a transition metal or a transition metal-containing compound;
the wavy bonds in formula (I), formula (II), and formula (III) represent connections to other portions of the binding link (where, e.g., the magnetic sensor is coupled to one wavy bond and the MNP is coupled to a different wavy bond); and
R in formula (I) and formula (III) is a hydrogen, unsubstituted hydrocarbyl, or substituted hydrocarbyl. An “unsubstituted hydrocarbyl” refers to a group that consists of hydrogen and carbon atoms only. A “substituted hydrocarbyl” refers to an unsubstituted hydrocarbyl in which at least one hydrogen of the unsubstituted hydrocarbyl has been substituted with at least one heteroatom or heteroatom-containing group, such as one or more elements from Group 13-17 of the periodic table of the elements.

The group represented by formula (I) is a carbamate moiety. The group represented by formula (II) is an ether moiety. The group represented by formula (III) is an amide moiety. Pd, Ru, Au, Pt, Cu, and Ru metals, or compounds comprising such metals, may break the C—N covalent bond present in the carbamate moiety of formula (I). Pd, Au, Pt, Cu, or compounds comprising such metals, may break the C—O covalent bond present in the ether moiety of formula (II). Pt, or compounds comprising Pt, may break the C—N covalent bond present in the amide moiety of formula (III).

Depending on, for example, chemical groups attached to the carbamate moiety, ether moiety, and amide moiety via the wavy bonds, the binding link is sensitive to a specific metal or specific metal-containing compound. For example, when a vinyl group (alkene) is two carbons away from an oxygen atom (beta to the oxygen atom), the C—N covalent bond of the carbamate moiety may be cleaved by Pd, Ru, or compounds comprising Pd or Ru, by the reaction shown in Scheme 3.

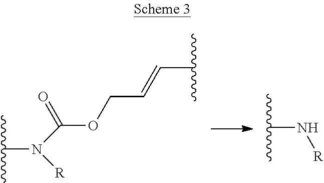

As another example, C—N covalent bond (when X is oxygen) of a carbamate moiety or the C—N covalent bond (when X is CH₂) of an amide moiety may be cleaved by Pt by the reaction shown in Scheme 4.

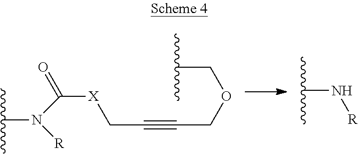

As another example, selected transition metals (TMs) may selectively break C—O covalent bonds of an ether moiety by varying the D as shown in Scheme 5.

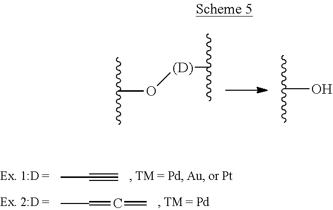

As shown in Example 1 of Scheme 5, the C—O covalent bond of an ether moiety may be broken by Pd, Au, or Pt, or a compound thereof, when D includes an alkyne. As shown in Example 2 of Scheme 5, the C—O covalent bond of an ether moiety may be selectively broken by Pd, or a compound thereof, when D includes a diene.

In action, for example, the analyte comprising a transition metal (or compound comprising a transition metal) may disrupt the binding link by breaking covalent bonds, e.g., C—N bond of a carbamate moiety, C—O bond of an ether moiety, and/or C—N bond of an amide moiety, present in the binding link, thereby releasing the MNP from the magnetic sensor.

### Detection of Formaldehyde and Phosphines

Embodiments described herein may be utilized to detect formaldehyde and phosphine analytes present in a sample. Formaldehyde may break a C—N covalent bond of a carbamate moiety present in a binding link (see formula (I)). Phosphines such as alkylphosphines (PR₃) may break a C—N covalent bond of a carbamate moiety present in a binding link (see formula (I)).

In action, for example, the formaldehyde or phosphine analyte may disrupt the binding link by breaking covalent bonds, e.g., C—N bond of a carbamate moiety, present in the binding link, thereby releasing the MNP from the magnetic sensor.

### Detection of Antibiotics

Embodiments described herein may be utilized to detect antibiotics present in a sample. Antibiotics may be used to break covalent bonds of peptides or used to break covalent bonds (e.g., solubilize) of cell membranes. Such a detection method may be utilized to screen various chemicals for antibiotic activity.

As an example, the binding link may include a lipid (which mimics a cell membrane). In action, for example, an antibiotic analyte may disrupt the lipid present in the binding link by breaking non-covalent bonds (solubilizing) present in the lipid, thereby releasing the MNP from the magnetic sensor. The antibiotic analyte may include a membrane-targeting antibiotic.

As another example, the binding link may include a peptidoglycan. D-Ala-D-Lac terminated peptidoglycan structures are found in vancomycin-resistant cell walls. Various small molecules having certain nucleophile-electrophile assemblies with complementary chirality to the peptidoglycan, may disrupt the D-Ala-D-Lac terminated peptidoglycan by breaking covalent bonds of the peptidoglycan, thereby releasing the MNP from the magnetic sensor. In this way, new antibiotics or chemicals to combat vancomycin resistance may be screened.

### Detection of Nucleases

Embodiments described herein may be utilized to detect nucleases present in a sample. Nucleases are hydrolytic enzymes that cleave covalent phosphodiester moieties between sugars and phosphate groups of nucleic acids. For example, bonds of 3′ to 5′ phosphodiester moieties and/or bonds of 5′ to 3′ phosphodiester moieties may be cleaved. Here, a phosphodiester moiety is formed between a 3rd carbon atom of one ribose sugar or deoxyribose sugar in one nucleotide and a 5th carbon atom of ribose sugar or deoxyribose sugar of the succeeding nucleotide (3′ to 5′ phosphodiester bond); or a phosphodiester bond is formed between a 5th carbon atom of ribose sugar or deoxyribose sugar of a nucleotide with a 3rd carbon atom of ribose or deoxyribose sugar of the preceding nucleotide.

For this example, the binding link may include a nucleic acid that contains a phosphodiester moiety, such as a DNA. In action, for example, the nuclease analyte may disrupt the binding link by breaking covalent bonds of the phosphodiester moiety present in the DNA, thereby releasing the MNP from the magnetic sensor.

### Detection of Proteases

Embodiments described herein may be utilized to detect proteases present in a sample. Proteases cleave covalent bonds present in polypeptides (e.g., proteins). Various proteases exist and include serine proteases, cysteine proteases, threonine proteases, aspartic proteases, glutamic proteases, metalloproteases, and asparagine peptide lyases. Proteolysis, the cleavage of peptide bond(s), may be specific, such that distinct proteases may be selectively detected.

For this example, the analyte includes a protease and the binding link includes a molecule containing a peptide bond, such as a polypeptide. In action, for example, the protease analyte disrupts the binding link by breaking covalent bond(s) (peptide bond(s)) present in the binding link, thereby releasing the MNP from the magnetic sensor.

### Detection of Reactive Oxygen Species

Embodiments described herein may be utilized to detect reactive oxygen species (ROS) present in a sample. ROS may include, but is not limited to, hydrogen peroxide, organic peroxides, or combinations thereof. For this example, the binding link may include a nucleic acid (e.g., a DNA or a ribonucleic acid (RNA)), a lipid, a polypeptide (e.g., protein), or combinations thereof. ROS reacts with nitrogenous bases (such as those present in DNA-adenine, guanine, cytosine, and thymine—or those present in RNA—adenine, guanine, cytosine, and uracil) resulting in nitrogenous base oxidation. ROS reacts with lipids, such as polyunsaturated fatty acids (membrane phospholipids) resulting in lipid peroxidation. ROS reacts with various bonds present in polypeptides including C—C bonds and/or C—N bonds.

By including materials that are reactive to ROS—e.g., a nucleic acid, a lipid, a polypeptide, or combinations thereof—in a binding link, ROS analytes may be detected. In action, for example, the ROS analyte may disrupt the binding link by breaking covalent bond(s) present in the nucleic acid, the lipid, or the polypeptide, thereby releasing the MNP from the magnetic sensor.

Various functional groups, such as disulfides, diselenides, thioketals, boronic acids, boronic esters, vinyldithiothers, aryloxalate esters, or proline oligomers, are efficiently cleaved (breaking covalent bonds) by ROS. Also, hydrophobic-hydrophilic transitions (breaking non-covalent bonds) may be induced by ROS interacting with moieties responsive to oxidative stress, such as thioethers, selenoethers, telluroethers. One or more of these functional groups may be integrated into binding links. For example, a disulfide moiety, diselenide moiety, boronic acid moiety, boronic ester moiety, vinyldithiother moiety, aryloxalate ester moiety, or proline oligomer moiety, thioether moiety, selenoether moiety, telluroether moiety, or combinations thereof may be integrated into a polymer or a block copolymer.

The polymer or block copolymer having one or more of the aforementioned moieties may then be used as a binding link to attach the MNP to the magnetic sensor. In action, for example, the ROS analyte disrupts the binding link by breaking covalent bond(s) and/or non-covalent bond(s) present in the functional group (e.g., disulfide bond) of the binding link, thereby releasing the MNP from the magnetic sensor.

Additionally, or alternatively, the polymer or block copolymer having one or more of the aforementioned moieties may be formed into a responsive hydrogel on the surface of the magnetic sensor, and inside the hydrogel is an MNP. In action, for example, the ROS analyte disrupts the binding link by breaking covalent bond(s) and/or non-covalent bond(s) present in the functional group (e.g., a diselenide bond) of the binding link, thereby releasing the MNP from the magnetic sensor.

The range of concentrations of ROS may be too low to be detected by direct detection. In some embodiments, chemical amplification (a form of indirect detection) may be used. For example, chemical amplification of peroxy radicals (PeRCA) may convert peroxy radicals present in the sample to NO₂ through an amplifying chain reaction that increases the concentration of NO₂ by, e.g., a factor of 100 to 200 compared to that of the peroxy radical. Organic peroxides, such as hydroxyl, alkoxyl, hydoperoxyl, or alkylperoxyl radicals are also converted to HO₂ through a two-step process. The overall chain reaction is given by Scheme 6.

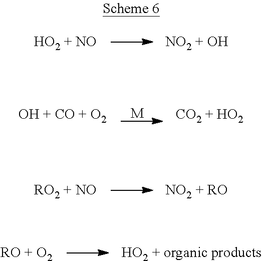

wherein: HO₂ is hydroperoxyl radical; NO is nitric oxide; NO₂ is nitrogen dioxide; OH is hydroxyl; CO is carbon monoxide; O₂ is molecular oxygen; CO₂ is carbon dioxide; M is a third body (e.g., an atom or molecule that stabilizes the reaction by taking energy away from the reactants); RO₂ is peroxy radical or organic peroxide, for example a n-propyl peroxy radical or i-propyl peroxy radical or a acyl peroxy or hydroperoxy acyl peroxy radical; RO is a reaction product from converting the organic peroxide; and organic products is a reaction product from converting the organic peroxide.

For amplifying the ROS, one or more additional materials are added to the sample. The one or more additional materials may include NO, O₂, and CO. Optionally, ethane or acetaldehyde may be used in place of CO. These materials, which may be in the form of gases, may be introduced to the apparatus for magnetically detecting the presence of a target analyte using known methods or may be introduced to the sample using known methods.

NO₂ reacts with various materials such as primary and/or secondary amines present in polypeptides (e.g., proteins), causing cleavage of peptide bond(s). In some embodiments, the binding link may include a polypeptide (or protein).

In action, for example, one or more additional materials (such as NO, O₂, and/or CO gases) are added to the sample being investigated for ROS. In the presence of the added one or more additional materials, the ROS is converted to NO₂ and the NO₂ disrupts the binding link (e.g., by cleaving covalent bonds present in the polypeptide of the binding link), thereby releasing the MNP from the magnetic sensor and indicating that a ROS is present in a sample. When ROS is not present in the sample, the added one or more additional materials will not form NO₂ and will not disrupt the binding link, indicating that ROS is not present in the sample.

### Detection of Topoisomerases

Embodiments described herein may be utilized to detect a toposiomerase present in a sample. Topoisomerases cleave covalent bonds present in nucleic acids. For this example, the binding link may include a nucleic acid such as DNA. In action, for example, the topoisomerase analyte may disrupt the binding link by breaking covalent bond(s) present in the nucleic acid, thereby releasing the MNP from the magnetic sensor.

### Detection of Topoisomerase Inhibitors and Protease Inhibitors

Embodiments described herein may be utilized to detect a toposiomerase inhibitor or a protease inhibitor present in a sample. These analytes may be tested by indirect detection.

For example, and with respect to topoisomerase inhibitors, topoisomerase may be added to a sample being investigated for topoisomerase inhibition and the binding link may include a nucleic acid such as a DNA. In the presence of the added topoisomerase (which is a material added to the sample), the topoisomerase inhibitor analyte may prevent topoisomerase from disrupting the binding link (e.g., by cleaving covalent bonds present in the nucleic acid of the binding link), thereby inhibiting release of the MNP from the magnetic sensor and indicating that a topoisomerase inhibitor is present in a sample. When topoisomerase inhibitor is not present in the sample, the added topoisomerase may disrupt the binding link by cleaving covalent bonds present in the nucleic acid of the binding link, indicating that a topoisomerase inhibitor is not present in the sample. Examples of topoisomerase inhibitors that may be tested include, but are not limited to, aminocoumarins, quinolones, anthracyclines, and camptothecin derivatives.

With respect to protease inhibitors, a protease, such as those described herein, may be added to a sample being investigated for protease inhibition and the binding link may include a polypeptide (e.g., a protein). In the presence of the added protease (a material added to the sample), the protease inhibitor analyte may prevent protease from disrupting the binding link (e.g., by cleaving covalent bonds present in the polypeptide of the binding link), indicating that a protease inhibitor is present in a sample. When protease inhibitor is not present in the sample, the added protease may disrupt the binding link by cleaving covalent bonds present in the polypeptide of the binding link, indicating that a protease inhibitor is not present in the sample.

### Detection of Antigens

Embodiments described herein may be utilized to detect an antigen or an antibody present in a sample. The binding link may include an antibody, an antigen, or combinations thereof. MNPs may be labeled with antibodies.

For example, the antibodies may be conjugated to the surface of the MNPs through their fragment crystallizable regions in such a way that their fragment antigen-binding regions are available for antigen recognition. As an example, the surface of the MNPs may be functionalized to contain primary amine groups, to which antibodies containing carboxylic acid groups may be covalently bound. The corresponding binding antigen, which may e.g. be a protein, a peptide, a polysaccharide, a lipid, or a nucleic acid, may be attached to the surface of the magnetic sensor for example by a covalent chemical bond. For example, the antigen may contain thiol groups, and the surface of the sensor may contain gold, to which thiol groups bind. In action, for example, an antigen analyte may disrupt the antibody-containing binding link, thereby releasing the magnetic nanoparticles from the surface. The disruption may be a result of breaking covalent bonds, non-covalent bonds, or combinations thereof.

As another example, the analyte may include an antigen (Ag′) or an antibody (Ab′) which binds to the corresponding antibody (Ab) or antigen (Ag) bonded to the MNP or the surface of the magnetic sensor. These Ag′ or Ab′ analytes may competitively displace corresponding molecules in the binding link between the magnetic sensor and the MNP as shown in Scheme 7. Scheme 7 shows competitive displacement where the analyte is antibody Ab′.

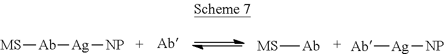

wherein: MS refers to magnetic sensor; Ab refers to a first portion of the bind link (the antibody); Ag refers to a second portion of the binding link (the antigen); the binding link is Ab-Ag; NP refers to magnetic nanoparticle; Ab′ refers to the analyte antibody.

As shown in Scheme 7, and prior to running the sample, the magnetic sensor is coupled to the MNP by the binding link Ab-Ag (shown as MS-Ab-Ag-NP). The Ab′ analyte competitively displaces the Ab of the binding link. According to the law of mass action the rate of a chemical reaction is directly proportional to the product of the activities of the reactants. As the analyte Ab′ is added, less Ab binds to Ag. Therefore the presence of Ab′ reduces the amount of MNPs bound to the surface. Overall, the reaction shown in Scheme 7 may disrupt the binding link, thereby releasing the MNP from the magnetic sensor by competitive displacement.

### Detection of CRISPR-Associated Proteins

Embodiments described herein may be utilized to detect clustered regularly interspaced short palindromic repeats (CRISPR)-Cas (CRISPR-associated proteins) present in a sample. As an example CRISPR-associated protein, Cas9 is an enzyme that uses CRISPR sequences as a guide to recognize and open up specific strands of DNA that are complementary to the CRISPR sequence.

For this example, the binding link may include a nucleic acid such as DNA. In action, for example, the CRISPR-Cas analyte may disrupt the binding link by breaking covalent bond(s) present in the DNA, thereby releasing the MNP from the magnetic sensor.

### Detection of Anti-CRISPR Proteins

Embodiments described herein may be utilized to detect anti-CRISPR proteins (Acrs) present in a sample. Acrs are small proteins, typically less than 200 amino acids, which inhibit the cleaving action of CRISPR-Cas. Examples of Acr proteins that may be detected include, but are not limited to, AcrE1, AcrE2, AcrE3, AcrE4, AcrF1, AcrF2, AcrF3, AcrF4, AcrF5, AcrF6, AcrF7, AcrF8, AcrF9, AcrF10, AcrIIA1, AcrIIA2, AcrIIA3, AcrIIA3, AcrIIC1, AcrIIC2, and AcrIIC3. These Acr proteins are known to inhibit the activity of nucleic acids such as DNA.

These Acr protein analytes may be tested by indirect detection. One or more additional materials may be added to the sample such as a Cas protein which would normally disrupt the nucleic acid in the binding link.

For example, Cas protein may be added to a sample being investigated for anti-CRISPR proteins and the binding link may include a nucleic acid such as DNA. In the presence of the added Cas protein (which is a material added to the sample), the Acr protein analyte may prevent Cas protein from disrupting the nucleic acid-containing binding link, thereby inhibiting release of the MNP from the magnetic sensor and indicating that an Acr protein is present in the sample. When Acr protein is not present in the sample, the added Cas protein may disrupt the binding link by cleaving covalent bonds present in the nucleic acid of the binding link, thereby releasing MNP from the magnetic sensor and indicating that a Acr protein is not present in the sample.

### Chemical Amplification

Chemical amplification may include chemical reactions that generate a higher concentration or amount of a species (e.g., a molecule), or a chemical reaction that generates a new species. The higher concentration or amount of species, or the new species, may disrupt the binding link, thereby releasing the MNP from the magnetic sensor. As described herein, embodiments of the present disclosure enable detection of target analytes present in a sample even at low concentrations. As also described herein, embodiments of the present disclosure enable indirect detection of target analytes present in a sample.

As an example, a chemical amplification reaction may be a reaction that generates a species that catalyzes another reaction and optionally also the succeeding catalyzed reaction. Such a reaction is seen with, e.g., the determination of iodide through its catalytic effect on the reaction between ceric (Ce(IV)) and arsenious (As(III)) ions or the estimation of lactate dehydrogenase by its catalytic action converting pyruvate to lactate.

An example of a chemical amplification reaction is one which replaces the conventional reaction used in a particular determination so that a more favorable measurement may be made. This may be repeated to provide a further favorable increase in measurement. Another example of a chemical amplification reaction might be a multiplication reaction, in which a given quantity of a species to be determined is converted into a multiple of said quantity by a cycle of chemical reactions.

Examples of chemical amplification reaction may include polymerase chain reaction (PCR), the Mohr or Leipert method, the PERCA (PEroxy Radical Chemical Amplification) technique, the lysis of an analyte labeled liposome by an antibody with the subsequent release of a larger amount of a substance previously contained inside of the liposome.

Embodiments described herein may be used with any suitable chemical amplification technique, such as those chemical amplification techniques described herein. Materials that may be added to samples for chemical amplification may include, but are not limited to, reactant, a reagent, an enzyme, a primer, an amplifier, an inhibitor, or combinations thereof.

Various chemical amplification methods are described herein. Besides these, other chemical amplification methods include DNA amplification and RNA amplification

### 1. DNA Amplification

In some embodiments, which may be combined with other embodiments, amplification may include nucleic acid amplification. Here, a sample containing a target to be detected may be mixed with one or more materials, such as a reactant, a reagent, an enzyme, a primer, an amplifier, an inhibitor, optionally other materials, or combinations thereof, and the resultant mixture may be introduced into the apparatus.

Amplification may be carried out using loop-mediated isothermal amplification (LAMP). Here, primers, polymerase, and optional materials may be introduced to the sample being tested for the presence of DNA (the target to be detected). Primers are short nucleic acids that conjugate to the target nucleic acid and provide a starting point for polymerase enzyme to add further nucleotides. The primers are specific to the DNA that is to be amplified and are designed individually by standard methods in the art. Selectivity of the amplification is achieved by designing the primers such that they specifically bind to the desired target DNA.

For example, two or more sets of primers, a temperature-stable strand displacing polymerase (for example, the Klenow fragment of Bacillus stearothermophilus (Bst) polymerase, such as Lyo-ready Bst DNA polymerase), and additional optional materials may be added to the sample. The additional optional materials may include deoxynucleotide triphosphates, which may contain deoxyadenosine triphosphate, deoxyguanosine triphosphate, deoxythymidine triphosphate, and deoxycytidine triphosphate as well as optional compounds such as cyclic GMP sodium (cGMP) salt, spermidine, dibasic sodium pyrophosphate, dithiothreitol, a deoxyribonuclease inhibitor, such as deoxyribonuclease (DNase). Other optional materials may include buffers, salts, acids or bases, chelating agents or other compounds to stabilize DNA, such as tris(hydroxymethyl)aminomethane, (4-(2-hydroxyethyl)-1-piperazinecthanesulfonic acid), potassium chloride, sodium chloride, calcium chloride, magnesium chloride ammonium sulfate, hydrochloric acid, sodium citrate, potassium hydroxide, magnesium sulfate, ethylenediaminetetraacetic acid, ethylene glycol-bis(β-aminoethyl ether)-N,N,N′,N′-tetraacetic acid, nonyl phenoxypolyethoxylethanol, 2-[4-(2,4,4-trimethylpentan-2-yl)phenoxy]ethanol, and proteases. These materials are commercially available or may be made by known methods. A person of ordinary skill would understand whether to use optional material(s) and what to use as the optional material(s).

Other methods of nucleic acid (analyte) amplification may be used, including, but not limited to rolling circle amplification (RCA), whole genome amplification (WGA), hybrid capture amplification, multiple displacement amplification (MDA), recombinase polymerase amplification (RPA), helicase-dependent amplification (HAD), nucleic acid-based amplification (NASBA), transcription-mediated amplification (TMA), strand displacement amplification (SDA), exponential amplification reaction (EXPAR), polymerase chain reaction (PCR), among others. Persons of ordinary skill would understand what nucleic acid amplification method may be used and what additional materials are to be added to the samples.

In some embodiments, DNA amplification techniques are used to amplify a reaction intermediate. The reaction intermediate may disrupt a binding link and release MNP from the magnetic sensor. Additionally, or alternatively, the reaction intermediate may be involved in one or more chemical reactions to produce a reaction product, and the reaction product disrupts the binding link and release MNP from the magnetic sensor.

By whichever mechanism, DNA amplification is leveraged by embodiments described herein. For example, LAMP or EXPAR may be utilized to multiply the DNA by, e.g., 10⁶. The amplified DNA may disrupt the binding link by, for example, competitive displacement and/or enzyme activation, among disruptions.

### 1.A. DNA Amplification: Competitive Displacement

In a non-limiting example, for small amounts of DNA present in a sample, the addition of primers, enzymes, and other optional materials allow DNA to be directly amplified. Following amplification of the DNA, the target DNA may be detected by competitive displacement of a DNA species. The binding link, positioned between the MNP and the magnetic sensor may include DNA. By amplifying the DNA, the large amount of DNA amplified displaces the DNA of the binding link, thereby releasing the MNP from the particle according to Scheme 8.

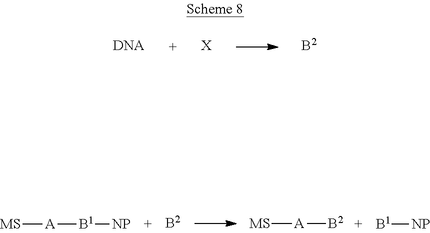

wherein: DNA refers to the unamplified DNA (the low concentration of DNA in the sample); X refers to amplifier; B² refers to the amplified DNA (higher concentration of DNA); MS refers to magnetic sensor; A refers to a first portion of the binding link; B¹ refers to a second portion of the binding link and includes the DNA of the binding link; and NP refers to magnetic nanoparticle.

As shown in Scheme 8, and prior to running the sample, the magnetic sensor is coupled to the MNP by the binding link A-B¹ (shown as MS-A-B¹-NP). Amplification of the DNA causes generation of high concentrations of DNA (B²). The high concentrations of B² disrupt the binding link by competitively displacing the B¹ present in the binding link, thereby releasing the MNP (e.g., in the form of B¹-NP) from the magnetic sensor. For competitive displacement, and in some embodiments, processes described herein may include introducing one or more materials that amplify the DNA as described herein to the sample. The one or more materials increase the concentration of the DNA in the sample from a first concentration (a low concentration) to a second concentration of the DNA (a high concentration, e.g., amplified DNA, B²). The binding link A-B¹ is adapted to be disrupted in the presence of the second concentration of the DNA, and the binding link A-B¹ may be adapted to be undisrupted in the presence of the first concentration of the DNA. The second concentration of the DNA causes competitive displacement of a nucleic acid present in the binding link upon introduction of the sample to the apparatus (e.g., the reaction shown in Scheme 8).

### 1.B. DNA Amplification: Disassociation of Nucleic Acid Strands

In some embodiments, which may be combined with other embodiments, the binding link may be formed by a segment of a nucleic acid with at least two strands with the one end of a first strand bound to the MNP and a second end of a second strand is bound to the sensor surface. Amplified DNA may hybridize to at least one of the nucleic acid strands, causing a disassociation between the first and the second strand and thus releasing the MNP from the sensor surface. The binding link in this example may include at least two strands. Nucleic acids having more than two strands are known. As an example, the binding link may include five single-stranded DNA strands associated together into a multistranded helix. In action, the amplified DNA disrupts the nucleic acid strands of the binding link by hybridizing with a strand of the binding link and disassociating the first and the second strand, resulting in release of the MNP from the magnetic sensor. In this example, the two or more strands may be non-covalently bonded to each other and the non-covalent bond may be broken by displacing at least one of the original strands.

In some variations, an additional material, such as an enzyme, which may be a helicase, such as E. coli UvrD or PcrA M6 helicase, or a polymerase, such as Bst polymerase, may be added, and the enzyme may be activated by the amplified DNA. The activated enzyme may then disrupt the binding link, resulting in release of the MNP from the magnetic sensor.

### 1.C. DNA Amplification: Enzyme Activation

Additionally, or alternatively, the amplified DNA may activate an enzyme present in the sample, and the enzyme disrupts the binding link. Here, for example, the DNA may be amplified as described above. An enzyme (E) such, as a protease (a proteolytic enzyme) may be activated by the higher amount of DNA to form DNA-E, and at high enough concentrations brought about by the amplification, sufficient amounts of DNA-E forms to cleave the A-D bond of the binding link, releasing the MNP from the magnetic sensor as shown in Scheme 9.

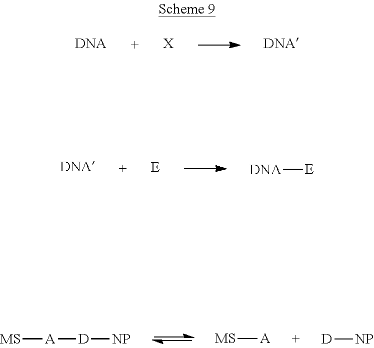

wherein: DNA refers to the unamplified DNA (the low concentration of DNA in the sample); X refers to amplifier; DNA′ refers to the amplified DNA (higher concentration of DNA); E refers to enzyme; DNA-E refers to DNA-activated enzyme; MS refers to magnetic sensor; A refers to a first portion of the binding link; D refers to a second portion of the binding link; NP refers to magnetic nanoparticle. The magnetic sensor is coupled to the MNP by the binding link A-D (shown as MS-A-D-NP).

As shown in Scheme 9, and prior to running the sample, the magnetic sensor is coupled to the MNP by the binding link A-D (shown as MS-A-D-NP). Amplification of the DNA causes generation of high concentrations of DNA (DNA′). The high concentrations of DNA′ activate the enzyme (E) and E disrupts the binding link by cleaving covalent bonds present in the binding link, thereby releasing the MNP (e.g., in the form of D-NP) from the magnetic sensor.

For enzyme activation, and in some embodiments, processes described herein may include introducing one or more materials that amplify the DNA as described herein to the sample. The one or more materials increase the concentration of the DNA in the sample from a first concentration (a low concentration) to a second concentration of the DNA (a high concentration, e.g., amplified DNA, DNA′). The binding link A-D is adapted to be disrupted in the presence of the second concentration of the DNA and the binding link A-D may be adapted to be undisrupted in the presence of the first concentration of the DNA. The second concentration of the DNA (the DNA′) causes activation of a proteolytic enzyme (E) present in the sample that breaks a covalent bond present in the binding link (illustrated by the breaking of the A-D bond) upon introduction of the sample to the apparatus (e.g., the reaction shown in Scheme 9).

Regardless of the method of DNA amplification, the amplified DNA may be detected by embodiments described herein. For example, the amplified DNA may disrupt a binding link, via breaking covalent bonds and/or non-covalent bonds present in the binding link, releasing a MNP from a magnetic sensor. Additionally, or alternatively, the amplified DNA may begin a cascade of events that result in a reaction product, and the reaction product disrupts the binding link via breaking covalent bonds and/or non-covalent bonds present in the binding link, releasing a MNP from a magnetic sensor.

### 2. RNA Amplification

In some embodiments, which may be combined with other embodiments, amplification may include nucleic acid amplification targeted to amplify RNA. Here, a sample containing a target to be detected may be mixed with various optional materials, such as a reactant, a reagent, an enzyme, a primer, an amplifier, an inhibitor, optionally other materials, or combinations thereof, and the resultant mixture may be introduced into the apparatus.

The amplification may be carried out by linear antisense RNA (aRNA) amplification. In a first round, and amplicon is formed by priming the analyte RNA followed by reverse transcription into double stranded complementary DNA (cDNA) using, for example, avian myeloblastoma virus (AMV) reverse transcriptase or Moloney murine leukemia virus reverse transcriptase, together with Ribonuclease H (RNase H) to split messenger RNA (mRNA) into random fragments that serve as primers for the cDNA generation by a polymerase, for example, Escherichia coli DNA polymerase I. The ends of the cDNA may be blunted by the enzyme T4 DNA polymerase. The cDNA is then amplified into antisense RNA via linear transcription by the enzyme T7 RNA polymerase after incorporating a promoter into the double stranded cDNA.

In a second round, the aRNA is primed with random primers followed by reverse transcription using, for example, AMV reverse transcriptase or Moloney murine leukemia virus reverse transcriptase. After priming with a T7 RNA polymerase promoter, e.g., oligo dT, a complementary cDNA strand is synthesized by using a DNA polymerase, e.g., a Klenow fragment T4 polymerase. The double-stranded DNA (dsDNA) is then transcribed into aRNA using a RNA polymerase, e.g. T7 RNA polymerase.

AMV reverse transcriptase, Moloney murine leukemia virus reverse transcriptase, RNase H, and polymerases such as Escherichia coli DNA polymerase I, T4 DNA polymerase, T7 RNA polymerase, Klenow fragment T4 polymerase, polymerase promoters such as oligo dT are available commercially or may be made by known methods.

Optional reagents for RNA amplification may include, but are not limited to, buffers, salts, acids or bases, chelating agents or other compounds to stabilize DNA, such as tris(hydroxymethyl)aminomethane, (4-(2-hydroxyethyl)-1-piperazineethanesulfonic acid), potassium chloride, sodium chloride, calcium chloride, magnesium chloride, ammonium sulfate, hydrochloric acid, sodium citrate, potassium hydroxide, magnesium sulfate, ethylenediaminetetraacetic acid, ethylene glycol-bis(β-aminoethyl ether)-N,N,N′,N′-tetraacetic acid, nonyl phenoxypolyethoxylethanol, 2-[4-(2,4,4-trimethylpentan-2-yl)phenoxy]ethanol, and proteases. together with a mixture of deoxynucleotide triphosphates, which may contain deoxyadenosine triphosphate, deoxyguanosine triphosphate, deoxythymidine triphosphate, and deoxycytidine triphosphate, guanosine 5′-triphosphate sodium salt, adenosine 5′-triphosphate disodium salt, uridine-5′-triphosphate, cytidine triphosphate, cyclic GMP sodium (cGMP) salt, spermidine, dibasic sodium pyrophosphate, dithiothreitol, a ribonuclease inhibitor, such as RNasin, SI nuclease. These materials are commercially available or may be made by known methods. A person of ordinary skill would understand whether to use optional material(s) and what to use as the optional material(s).

Amplification may be carried out by RNA isothermal co-assisted and coupled amplification (RICCA). Here, for example, the RNA analyte is primed with a T7-bearing anti-sense primer and a first strand of cDNA is synthesized using a reverse transcriptase, such as AMV reverse transcriptase. The DNA strand is cut by a RNase, e.g., RNase H, and the second strand of cDNA is synthesized by a DNA polymerase, and a sense primer. In two parallel cycles, the double strand (ds) cDNA is transcribed into aRNA by an RNA polymerase, for example T7 RNA polymerase, followed by priming and synthesis of a first strand of cDNA. After cutting the RNA strand by a RNase, the T7-bearing antisense primer binds to the first cDNA strand, thus completing the loop. In a parallel second loop, the double stranded DNA is amplified by adding a single-strand DNA-binding protein (SSB) and a recombinase enzyme. In a second step, a strand displacing DNA polymerase, for example Bst DNA polymerase, creates a double stranded DNA, thus closing the DNA amplification loop.

Optional reagents may include RNA stabilizing compounds, such as surfactants, such as nonyl phenoxypolyethoxylethanol, 2-[4-(2,4,4-trimethylpentan-2-yl)phenoxy]ethanol, chelating agents, such as ethylenediaminetetraacetic acid, ethylene glycol-bis(β-aminoethyl ether)-N,N,N′,N′-tetraacetic acid, protein denaturants, such as guanidinium thiocyanate (GTC) or guanidinium isothiocyanate, or proteases; buffers, which may contain disodium hydrogen phosphate, potassium dihydrogen phosphate, tris(hydroxymethyl)aminomethane, hydrochloric acid, sodium hydroxide, potassium hydroxide, potassium chloride, sodium chloride, magnesium chloride. These materials are commercially available or may be made by known methods. A person of ordinary skill would understand whether to use optional material(s) and what to use as the optional material(s).

Additionally, or alternatively, RNA may be reverse transcribed to complementary DNA using a reverse transcriptase enzyme (RTase), for example Moloney murine leukemia virus RTase, or avian myeloblastosis virus RTase. The resulting DNA may then be detected using the methods described in the DNA amplification Section or by other DNA amplification methods.

Additionally, or alternatively, RNA may be directly amplified by nucleic acid-based amplification (NASBA), rolling circle replication (RCR), or recombinase polymerase amplification (RPA). A person of ordinary skill in the art would know how to perform and what materials to utilize for such RNA direct amplification methods.

First example of RNA amplification: In the first round, first-strand cDNA synthesis by reverse transcription (RT) is primed from mRNA after oligo (dT)-T7 primer anneals to the poly(A) tail of mRNA. RNase H is then used to digest portions of the bound mRNA to create RNA fragments that serve to prime second-strand cDNA by DNA polymerase (pol). Finally, aRNA is amplified via linear in vitro transcription by T7 RNA polymerase, using the T7 RNA polymerase promoter incorporated in the double-stranded cDNA. In the second round, first-strand synthesis is primed by random primers instead of the oligo (dT)-T7 primer by reverse transcriptase using the aRNA as a template instead of mRNA. After RNA denaturation, second-strand synthesis is primed with the oligo (dT)-T7 primer, which binds to the poly(A) tail of the cDNA created during first-strand synthesis by DNA polymerase. Finally, RNA is again linearly amplified through the enzymatic activity of T7 RNA polymerase acting on its promoter that is incorporated into the double-stranded cDNA.

Second example of RNA amplification: The first step is the synthesis of an oligo (dT) primer that is extended at the 5′ end with a T7 RNA polymerase promoter. This oligonucleotide may be used to prime the poly(A) mRNA population for cDNA synthesis. After the first-strand cDNA is synthesized, the second-strand cDNA is made using either “RNA nicking and priming” for RNA in solution or “hairpinning” for tissue sections. This is followed by a brief SI nuclease treatment and “blunt-ending” with T4 DNA polymerase. The cDNA is now ready for amplification using the T7 RNA polymerase promoter to direct the synthesis of RNA. The RNA made using this technique is antisense to the poly(A)+RNA and may either be used as a probe or be cloned.

Third example of RNA amplification: AMV reverse transcriptase (available from Seikagaku) and oligo (dT)-T7 primer are included in the sample to facilitate cDNA synthesis. An electrode is attached to the sample for times ranging from 1 to 20 min. The cellular contents are then aspirated into the patch pipette with a slight amount of suction. cDNA synthesis is continued in the patch electrode at 37° C. for 60 min. Single-strand cDNA synthesis is followed by ethanol precipitation. The precipitated DNA is dissolved in water and heated at 95° C. for 3 min. Second-strand cDNA synthesis is accomplished with the modification that 10 units each of T4 DNA polymerase and Klenow is used in the synthesis. After SI nuclease treatment, end repair, and ethanol precipitation, the double-stranded cDNA is dissolved in Tris/EDTA (TE) and drop-dialyzed for 4 hr. The DNA is recovered from the filter, and aRNA is made from DNA sample in two batches. The T7 RNA polymerase used in this reaction may be in any suitable concentration. After phenol/chloroform extraction and ethanol precipitation, the aRNA is reamplified by dissolving the pellet in water, adding 10-100 ng of random hexanucleotide primers (this may vary depending on yield of aRNA), a buffer (e.g., 500 mM Tris, pH 8.3, 1.2 M KCl, 100 mM MgCl₂, 0.5 mM sodium phosphate), dithiothreitol (DTT), dNTPs, RNase, and reverse transcriptase, and incubating at 37° C. for 1 hr. This mixture is phenol/chloroform extracted and ethanol precipitated. The pellet is dissolved in water and is heat denatured at 95° C. for 2 hours.

Regardless of the method of RNA amplification, the amplified RNA may be detected by embodiments described herein. For example, the amplified RNA may disrupt a binding link, via breaking covalent bonds and/or non-covalent bonds present in the binding link, releasing a MNP from a magnetic sensor. Additionally, or alternatively, the amplified RNA may begin a cascade of events that result in a reaction product, and the reaction product disrupts the binding link via breaking covalent bonds and/or non-covalent bonds present in the binding link, releasing a MNP from a magnetic sensor.

### Structure-Switching Aptamers

Structure-switching aptamers may be utilized with embodiments of the present disclosure. Aptamers are oligomers of artificial single-stranded DNA (ssDNA), RNA, or xeno nucleic acids (XNA). Structure-switching aptamers are aptamers that switch its structure upon binding with a target analyte. Structure-switching aptamers may be utilized with any suitable target analyte to be detected such as proteins, small molecules, metal ions, bacterial cells, viruses, cancer cells, tissues, antibiotics, or toxins, among others.

A structure-switching aptamer(s) may be added to the sample prior to introducing the sample to the apparatus. In action, the structure-switching aptamer may bind the target analyte, and upon binding, may trigger chemical amplification, such as those described herein. The species that is amplified may disrupt a binding link via breaking covalent bonds and/or non-covalent bonds present in the binding link, releasing a MNP from a magnetic sensor, Additionally, or alternatively, the binding of the target analyte with the structure-switching aptamer may begin a cascade of events that result in a reaction product. The reaction product disrupts the binding link via breaking covalent bonds and/or non-covalent bonds present in the binding link, releasing a MNP from a magnetic sensor.

As is apparent from the foregoing general description and the specific aspects, while forms of the aspects have been illustrated and described, various modifications may be made without departing from the spirit and scope of the present disclosure. Accordingly, it is not intended that the present disclosure be limited thereby. Likewise, the term “comprising” is considered synonymous with the term “including.” Likewise whenever a composition, an element, a group of elements, or a method is preceded with the transitional phrase “comprising,” it is understood that we also contemplate the same composition, method. or group of elements with transitional phrases “consisting essentially of,” “consisting of,” “selected from the group of consisting of,” or “Is” preceding the recitation of the composition, element, elements, or method, and vice versa, such as the terms “comprising,” “consisting essentially of,” “consisting of” also include the product of the combinations of elements listed after the term.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 5 includes the numbers 1, 1.5, 2, 2.75, 3, 3.80, 4, 5, among other numbers. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, aspects comprising “a magnetic sensor” includes aspects comprising one, two, or more magnetic sensors, unless specified to the contrary or the context clearly indicates only one magnetic sensor is included.

While the foregoing is directed to aspects of the present disclosure, other and further aspects of the disclosure may be devised without departing from the basic scope thereof, and the scope thereof is determined by the claims that follow.

What is claimed is:

1. An apparatus for magnetically detecting the presence of a target analyte in a sample, the apparatus comprising:
a plurality of magnetic nanoparticles; and
a plurality of magnetic sensors disposed within a fluidic channel, each magnetic sensor of the plurality of magnetic sensors coupled, by a binding link, to at least one magnetic nanoparticle of the plurality of magnetic nanoparticles, the binding link adapted to be disrupted in the presence of a target analyte that breaks a covalent bond of the binding link to release the at least one magnetic nanoparticle from the magnetic sensor and indicate the presence of the target analyte.

2. The apparatus of claim 1, further comprising processing circuitry configured to detect a signal from the magnetic sensor to indicate the absence of the at least one magnetic nanoparticle when the binding link is disrupted, thereby indicating the presence of the target analyte.

3. The apparatus of claim 1, wherein the apparatus is adapted to detect release of the at least one magnetic nanoparticle at the time of the release of the at least one magnetic nanoparticle.

4. The apparatus of claim 1, wherein at least one binding link is adapted to be disrupted in the presence of a control analyte present in the sample, the control analyte different from the target analyte.

5. The apparatus of claim 1, wherein the binding link of the apparatus is adapted to be disrupted at a second concentration of the target analyte, the second concentration of the target analyte generated from a chemical amplification reaction on a first concentration of the target analyte.

6. The apparatus of claim 1, wherein the binding link of the apparatus is adapted to be disrupted in the presence of a species, the species generated from a chemical reaction on the target analyte.

7. The apparatus of claim 1, wherein the binding link comprises a nucleic acid, a polypeptide, a peptidoglycan, or combinations thereof.

8. The apparatus of claim 7, wherein:
when the binding link comprises the nucleic acid, the target analyte comprises an enzyme, a mutagenic chemical, a CRISPR-associated protein, or combinations thereof;
when the binding link comprises the polypeptide, the target analyte comprises an enzyme, a reactive oxygen species, or combinations thereof;
when the binding link comprises the peptidoglycan, the target analyte comprises an antibiotic; or
combinations thereof.

9. The apparatus of claim 8, wherein, when the binding link comprises the nucleic acid and the target analyte comprises the enzyme, the enzyme comprises a topoisomerase, a helicase, a polymerase, a nuclease, or combinations thereof.

10. The apparatus of claim 8, wherein, when the binding link comprises the polypeptide and the target analyte comprises the enzyme, the enzyme comprises a protease.

11. The apparatus of claim 1, wherein the binding link comprises a carbamate moiety, an amide moiety, an ether moiety, a phosphodiester moiety, or combinations thereof.

12. The apparatus of claim 11, wherein:
when the binding link comprises the carbamate moiety, the amide moiety, the ether moiety, or combinations thereof, the target analyte comprises a transition metal from Group 3 to Group 12 of the periodic table of the elements;
when the binding link comprises the carbamate moiety, the target analyte comprises formaldehyde, a phosphine, or combinations thereof;
when the binding link comprises the phosphodiester moiety, the target analyte comprises a nuclease; or
combinations thereof.

13. An apparatus for magnetically detecting the presence of a target analyte in a sample, the apparatus comprising:
a plurality of magnetic nanoparticles; and
a plurality of magnetic sensors disposed within a fluidic channel, each magnetic sensor of the plurality of magnetic sensors coupled, by a binding link, to at least one magnetic nanoparticle of the plurality of magnetic nanoparticles, the binding link adapted to be disrupted in the presence of a target analyte that breaks a non-covalent bond of the binding link to release the at least one magnetic nanoparticle from the magnetic sensor and indicate the presence of the target analyte.

14. The apparatus of claim 13, further comprising processing circuitry configured to detect a signal from the magnetic sensor to indicate the absence of the at least one magnetic nanoparticle when the binding link is disrupted, thereby indicating the presence of the target analyte.

15. The apparatus of claim 13, wherein the apparatus is adapted to detect release of the at least one magnetic nanoparticle at the time of the release of the at least one magnetic nanoparticle.

16. The apparatus of claim 13, wherein at least one binding link is adapted to be disrupted in the presence of a control analyte present in the sample, the control analyte different from the target analyte.

17. The apparatus of claim 13, wherein the binding link of the apparatus is adapted to be disrupted at a second concentration of the target analyte, the second concentration of the target analyte generated from a chemical amplification reaction on a first concentration of the target analyte.

18. The apparatus of claim 13, wherein the binding link of the apparatus is adapted to be disrupted in the presence of a species, the species generated from a chemical reaction on the target analyte.

19. The apparatus of claim 13, wherein the binding link comprises a lipid, a nonpolar molecule, oil, wax, grease, streptavidin-biotin, a polymer, a salt, or combinations thereof.

20. The apparatus of claim 19, wherein:
when the binding link comprises the lipid, the target analyte comprises an antibiotic;
when the binding link comprises the nonpolar molecule, the oil, the wax, the grease, or combinations thereof, the target analyte comprises a surfactant;
when the binding link comprises the streptavidin-biotin, the target analyte comprises a solvent having a lower polarity than water;
when the binding link comprises the polymer, the target analyte comprises a solvent that solubilizes the polymer;
when the binding link comprises the salt, the target analyte comprises an ionic liquid; or
combinations thereof.

21. The apparatus of claim 13, wherein the binding link comprises two or more first nucleic acids non-covalently bonded to one another.

22. The apparatus of claim 21, wherein the target analyte comprises an enzyme, a second nucleic acid, or combinations thereof, the second nucleic acid adapted to displace at least one first nucleic acid of the two or more first nucleic acids.

23. A process for magnetically detecting the presence of a target analyte in a sample, the process comprising:
introducing a sample to an apparatus for magnetically detecting the presence of a target analyte in the sample; and
determining the presence of the target analyte by determining release of at least one magnetic nanoparticle from at least one magnetic sensor of the apparatus at the time of the release of the at least one magnetic nanoparticle.

24. The process of claim 23, wherein the apparatus for magnetically detecting the presence of the target analyte comprises:
a plurality of magnetic nanoparticles; and
a plurality of magnetic sensors disposed within a fluidic channel, each magnetic sensor of the plurality of magnetic sensors coupled, by a binding link, to at least one magnetic nanoparticle of the plurality of magnetic nanoparticles, the binding link adapted to be disrupted in the presence of the target analyte that breaks a covalent bond, a non-covalent bond, or both of the binding link to release the at least one magnetic nanoparticle from the magnetic sensor and indicate the presence of the target analyte.

25. The process of claim 24, wherein the apparatus further comprises processing circuitry configured to detect a signal from the magnetic sensor to indicate the absence of the at least one magnetic nanoparticle when the binding link is disrupted, thereby indicating the presence of the target analyte.

26. The process of claim 23, wherein the determining the presence of the target analyte comprises:
measuring a value of a characteristic of the magnetic sensor after introducing the sample; and
determining a change in the value of the characteristic by comparing the measured value of the characteristic to a baseline value of the characteristic.

27. The process of claim 23, wherein, prior to introducing the sample, the process further comprises:
introducing one or more materials to the sample, the one or more materials adapted to:
increase a concentration of the target analyte in the sample from a first concentration of the target analyte to a second concentration of the target analyte, the binding link adapted to be disrupted by the second concentration of the target analyte;
generate a species from the target analyte, the species different from the target analyte, the binding link adapted to be disrupted by the species; or
combinations thereof.

28. The process of claim 23, wherein:
the target analyte comprises a nucleic acid; and
prior to introducing the sample, the process further comprises introducing one or more materials to the sample, the one or more materials adapted to increase a concentration of the nucleic acid in the sample from a first concentration of the nucleic acid to a second concentration of the nucleic acid.

29. The process of claim 28, wherein the second concentration of the nucleic acid causes activation of an enzyme present in the sample that breaks a covalent bond present in the binding link after introducing the sample to the apparatus.

30. The process of claim 28, wherein the second concentration of the nucleic acid causes competitive displacement of a nucleic acid present in the binding link after introducing the sample to the apparatus.

31. The process of claim 28, wherein the second concentration of the nucleic acid causes disassociation of a nucleic acid strand present in the binding link after introducing the sample to the apparatus.
